(19) United States
(12) Patent Application Publication
(10) Pub. No.: US 2024/0366214 A1
(43) Pub. Date: Nov. 7, 2024

(54) DEVICES, SYSTEMS, AND METHODS FOR ARTICULATION AND/OR RETROFLEXION OF A MEDICAL DEVICE

(71) Applicant: Boston Scientific Scimed, Inc., Maple Grove, MN (US)

(72) Inventors: John Mims, Austin, TX (US); Jason N. Mehta, Austin, TX (US); Ryan Gilkey, Kyle, TX (US)

(73) Assignee: Boston Scientific Scimed, Inc., Maple Grove, MN (US)

(21) Appl. No.: 18/655,065

(22) Filed: May 3, 2024

Related U.S. Application Data
(60) Provisional application No. 63/541,152, filed on Sep. 28, 2023.
(60) Provisional application No. 63/464,007, filed on May 4, 2023.

(51) Int. Cl. A61B 17/04; A61B 17/00

(52) U.S. Cl. CPC A61B 17/0469; A61B 2017/00296; A61B 2017/00323; A61B 2017/00367; A61B 2017/00477; A61B 2017/047

(57) ABSTRACT

Systems and devices are provided with respect to an existing medical device to retroflex a flexible elongate member of the medical during in vivo use.

### CROSS-REFERENCE TO RELATED APPLICATIONS

This application claims the benefit of priority under 35 U.S.C. § 119 to U.S. Provisional Application No. 63/541,152, filed Sep. 28, 2023, and U.S. Provisional Application No. 63/464,007, filed May 4, 2023, the entire disclosures of which are hereby incorporated by reference herein for all purposes. Any and all priority claims identified in the Application Data Sheet, or any correction thereto, are hereby incorporated by reference under 37 C.F.R. § 1.57.

### FIELD

The present disclosure relates to devices, systems, and methods for effecting retroflexion of a medical device. In some embodiments, the devices and/or systems are mounted with respect to a pre-existing medical device, which may be steerable, to effect retroflexion, or to enhance already-provided retroflexion capabilities of the pre-existing medical device.

### BACKGROUND

Various medical scopes include various mechanism, such as internal pulleys and cables, to navigate the scope through a patient's body (including tortuous body passages). For instance, some medical scopes may be articulated in one or more planes, such as for up-down movement and/or left-right movement. Moreover, various endoscopic devices (e.g., instruments, tools, etc.) and systems for performing surgical operations or procedures are configured to be used with a medical cope, such as a pre-existing endoscope (e.g., a commercially available endoscope, or endoscope otherwise not customized for the devices or systems to be used therewith). Medical scopes typically include one or more working channels through which one or more devices may be advanced to or retracted from a target site within a patient's body. Various endoscopic devices that are designed to be delivered via a working channel of a scope (sometimes referred to as Through-the-Scope or “TTS” devices) are directed to a target site within a patient via flexing/articulation/bending of the scope. That is, as the target site is visualized by manipulation of the scope, the endoscopic device is translated longitudinally through the scope's working channel to be advanced to the target site. Medical scopes may also include various mechanisms configured and arranged to articulate a distal end of the scope, such as to direct devices extended through the scope's working channel to the desired target site within the patient's body. However, there are times when the ability of the scope to flex is limited either by the limits of maximum flexion of the scope, or by diminished performance of the scope over time. For instance, the inner workings of the scope used for navigation and articulation may become worn and/or cables stretched from repeated use and re-sterilization, such that performance thereof lags over time. Moreover, the bulk added by various devices extended through or along the scope may also affect articulation of the scope. There are times when a user of a scope can visualize an anatomical area which they wish to access, but the scope is unable to articulate sufficiently either to be directed toward the target site or to facilitate advancement of another device to the target site. In some scope systems, additional devices and systems are used in conjunction with the scope, and may add bulk to the overall system. During use, it may be difficult to maneuver the scope as a result of constraints imposed by an external system coupled to the scope. In view of various challenges to articulating a scope as desired during various procedures, desired retroflexion may not always be accomplished as easily as possible. It is with respect to these and other considerations that the present improvements may be useful.

### SUMMARY OF THE DISCLOSURE

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a retroflexion device is disclosed for retroflexing a flexible elongate member of a medical device. The retroflexion device includes a distal mount configured to be mounted with respect to the distal end of the flexible elongate member; a pivot mount configured to be mounted with respect to the flexible elongate member proximal to the distal mount; and an elongated actuation component having a proximal end and a distal end with a length extended therebetween selected to extend along the length of the flexible elongate member. In some aspects, a proximal end of the actuation component is controllable along a proximal end of the flexible elongate member; a distal end of the actuation component is operatively associated with a distal end of the flexible elongate member via the distal mount; and an actuation region of the actuation component pivots with respect to the pivot mount when the actuation component is pulled proximally to cause the flexible elongate member to retroflex.

In some aspects, the retroflexion device further includes a flexible tubular elongate member having at least one lumen extending therethrough through which the actuation component extends. In some aspects, the flexible tubular elongate member is configured to be mounted along the flexible elongate member. In some aspects, the flexible tubular elongate member has a proximal end and a distal end defining a length therebetween selected to be less than the length of the flexible elongate member; the flexible tubular elongate member is configured to be mounted with respect to the flexible elongate member with the distal end of the flexible tubular elongate member proximal to the distal end of the flexible elongate member; and the distal end of the flexible tubular elongate member forms the pivot mount for the actuation component. In some aspects, the flexible tubular elongate member has more than one lumen extending therethrough to allow additional devices to be extended through the flexible tubular elongate member. In some aspects, the flexible tubular elongate member has a proximal end and a distal end; and the retroflexion device further comprises a proximal mount provided along the proximal end of the flexible tubular elongate member; and the proximal end of the actuation component is operatively associated with the proximal mount.

In some aspects, the retroflexion device further includes a proximal mount, the proximal end of the actuation component being operatively associated with the proximal mount. In some aspects, the retroflexion device further includes a pull handle on the proximal end of the actuation component. In some aspects, the proximal mount includes a pull handle mount. In some aspects, the retroflexion device further includes a locking system mounted on the proximal mount and configured to temporarily retain the actuation component under tension to retroflex the flexible elongate member.

In accordance with various principles of the present disclosure, a retroflexion system includes a medical device with a flexible elongate member having a proximal end and a distal end and an outer surface extending between the proximal end and the distal end; a flexible tubular elongate member having a proximal end and a distal end and defining a lumen therethrough, the flexible tubular elongate member configured to be mounted with respect to the outer surface of the flexible elongate member; an actuation component extending through the lumen through the flexible tubular elongate member and having a distal end operatively associated with the distal end of the flexible elongate member, and a proximal end accessible to pull the actuation component proximally to retroflex the distal end of the flexible elongate member.

In some aspects, the retroflexion system further includes a distal mount coupled to the distal end of the flexible elongate member, the distal end of the actuation component operatively associated with the distal end of the flexible elongate member via the distal mount; and a pivot mount mounted with respect to the flexible elongate member proximal to the distal mount and about which an actuation region of the actuation component pivots to retroflex the distal end of the flexible elongate member. In some aspects, the length of the flexible tubular elongate member is less than the length of the flexible elongate member; the flexible tubular elongate member is mounted with respect to the flexible elongate member with the distal end of the flexible tubular elongate member proximal to the distal end of the flexible elongate member; and the distal end of the flexible tubular elongate member defines the pivot mount.

In some aspects, the retroflexion system further includes a proximal mount provided along the proximal end of the flexible elongate member and operatively associated with the proximal end of the flexible elongate member. In some aspects, the proximal end of the actuation component is operatively associated with the proximal mount. In some aspects, the retroflexion system further comprising a pull handle at a proximal end of the actuation component, wherein the proximal mount comprises a mount for the pull handle and a locking system configured to temporarily retain the actuation component under tension to retroflex the flexible elongate member.

In some aspects, the medical device is an endoscope.

In accordance with various principles of the present disclosure, method of retroflexing a distal end of a flexible elongate member of a medical device includes extending an elongated actuation component along the exterior of the flexible elongate member and fixing a distal end of the actuation component with respect to a distal end of the flexible elongate member; and pulling the actuation component proximally to cause the actuation component to pivot about a pivot mount proximal to the distal end of the actuation component and the distal end of the flexible elongate member to cause the flexible elongate member to retroflex.

In some aspects, pulling the actuation component includes pulling the proximal end of the actuation component adjacent a proximal end of the flexible elongate member. In some aspects, the method further includes securing a portion of the actuation component with respect to a locking system to hold the flexible elongate member in a retroflexed configuration.

In some aspects, the method further includes retroflexing the distal end of the flexible elongate member greater than 180°.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 is a schematic illustration of an example of an embodiment of a retroflexion device system formed in accordance with various principles of the present disclosure.

FIG. 1A is a detail view of the encircled area in FIG. 1.

FIG. 2 is a perspective view of an endoscopic suturing system according an embodiment of the invention.

FIG. 3 is a proximal perspective view of a suturing device of the endoscopic suturing system of FIG. 2.

FIG. 4 is a side elevation view of a suturing device, of indefinite length, of the endoscopic suturing system of FIG. 2.

FIG. 5 is a perspective distal end view of a cap assembly attached at the distal end of an endoscope of the endoscope suturing system.

FIG. 6 is a perspective distal end view of cap assembly of the suturing device relative to an endoscope for the endoscopic suturing system.

FIG. 7 is a cross section view across line VII-VII in FIG. 2.

FIG. 8 is a schematic distal end view of the cap assembly of the suturing device relative to an endoscope for the endoscopic suturing system.

FIG. 9 is a view similar to that of FIG. 8 with the endoscope rotated into alignment.

FIG. 10 is a view similar to that of FIG. 6 with the endoscope shown properly oriented and advanced with respect to the distal end of a cap assembly.

FIG. 11 is a schematic illustration of a tape applicator.

FIG. 12 is a schematic illustration of a taping system for securing a sheath of the suturing device relative to the endoscope.

FIG. 13 and FIG. 14 illustrate features of the tape of the tape system.

FIG. 15 and FIG. 16 are schematic illustrations of a retroflex system for the endoscopic system, as applied at the proximal handle of the system.

FIG. 17 is a schematic illustration of a detail of one embodiment of a lock for the retroflex system.

FIG. 18 is a schematic illustration of the endoscopic system subject to distal retroflex as applied by the retroflex system.

FIG. 19 is a schematic illustration of a detail of operation of the lock of FIG. 17.

FIGS. 20-24 illustrate use of the tape applicator to apply tape at the cap assembly to secure the cap assembly relative to the distal end of the endoscope.

FIG. 25 illustrates use of the tape along the sheath to secure the sheath to the endoscope.

FIGS. 26-30 illustrate use of the endoscopic system to endoscopically suture tissue.

FIG. 31 and FIG. 32 illustrate disassembly of the tape from about the cap assembly.

FIG. 33 illustrates disassembly of the sheath from the endoscope.

FIG. 34 and FIG. 35 illustrate examples of alignment of a cap assembly as in FIG. 8 and FIG. 9.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. As understood herein, “corresponding” is intended to convey a relationship between components, parts, elements, etc., configured to interact with or to have another intended relationship with one another. Finally, reference to “at” a location or site is intended to include at and/or about the vicinity of (e.g., along, adjacent, proximate, etc.) such location or site.

In accordance with various principles of the present disclosure, a retroflexion device and system are configured for mounting with respect to a flexible elongate member to articulate and retroflex the flexible elongate member. It will be appreciated that terms such as engage, receive, mount, couple, connect, attach, associate with, operatively associate with, etc., including other grammatical forms thereof, may be used interchangeably herein without intent to limit unless otherwise indicated. It will further be appreciated that, as used herein, retroflexion (including various grammatical forms thereof) is intended to refer to increased flexibility and bending of a device, such as to bend such device backwards, e.g., beyond 90° from its initial direction. In other words, a distal end of a generally longitudinally extending device extends distally, but may be retroflexed to extend in a proximal direction. The increased flexibility and degree of articulation which may be achieved by the flexible elongate member with the aid of a retroflexion device and system formed in accordance with various principles of the present disclosure allows increased access to anatomical sites within a body, particularly during transluminal and/or endoscopic procedures in which the patient's body is accessed in a minimally invasive manner (i.e., without cutting open the patient's body surgically). Moreover, a retroflexion device and system formed in accordance with various principles of the present disclosure imparts the flexible elongate member with which it is associated the ability to retroflex in a manner which may not otherwise be achievable. The increased articulation, including the ability to be retroflexed, allows the flexible elongate member greater access to the patient's anatomy than previously afforded or achievable by prior devices and systems. The flexible elongate member may be a flexible elongate member of a medical scope (e.g., the member or component of a medical scope which is inserted into patient, such an insertion member or insertion tube of the medical scope), and/or a member of another medical device that is to be retroflexed within a patient, the present disclosure not being limited in this regard.

In some aspects, a retroflexion device and system formed in accordance with various principles of the present disclosure are associated with a flexible elongate member in a manner allowing the retroflexion device and system to articulate and retroflex a distal end of the flexible elongate member. The device may be mounted with respect to the flexible elongate member as part of the system which includes additional components, including, without limitation, various mounts. The mounts include, without limitation, mounts configured to associate the retroflexion device and the flexible elongate member, mounts configured to retain a user engagement component of the retroflexion device, and mounts configured to guide an actuation component of the retroflexion device. In some aspects, the retroflexion system includes an auxiliary flexible elongate member defining a lumen through which at least the actuation component of the retroflexion device extends along the medical device to be flexed by the retroflexion device. The auxiliary flexible elongate member may define additional lumens, such as to provide auxiliary working channels supplementing the working channel of the flexible elongate member with respect to which the retroflexion device and system are associated.

In some aspects, a retroflexion device formed in accordance with various principles of the present disclosure is associated with a flexible elongate member which has a steering system capable of articulating at least a portion of the flexible elongate member. For instance, the retroflexion device may be configured to be associated with a medical scope capable of steering and articulation independently of the retroflexion device. A retroflexion device formed in accordance with various principles of the present disclosure may aid, supplement, alter, increase, etc., the articulation capabilities of the flexible elongate member. By being formed independently of the medical scope, the retroflexion device is capable of movements independent of the medical scope, and therefore may effectively eliminate effects of variability of the articulation of the medical scope (e.g., based on manufacturing specification, wear, or other alterations to the function which may occur with repeated use, etc.). A retroflexion device and system formed in accordance with various principles of the present disclosure allows the flexible elongate member with which it is associated to achieve more consistent flexing and/or a greater degree of flexing than previously achievable.

Various embodiments of devices and/or systems for effecting retroflexion of a medical device, and associated methods, will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

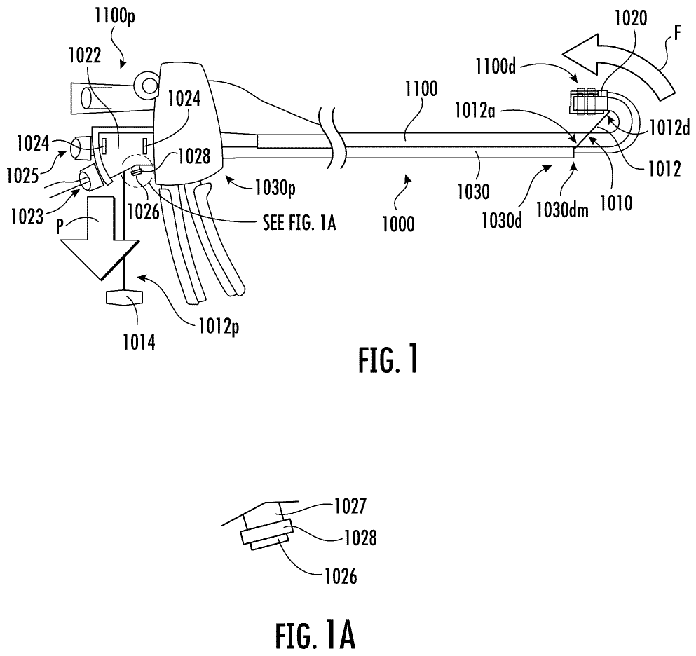

Turning now to the drawings, an example of an embodiment of a retroflexion system 1000 formed in accordance with various principles of the present disclosure is illustrated in FIG. 1. The illustrated example of an embodiment of a retroflexion system 1000 includes a retroflexion device 1010 operatively associated with a flexible elongate member 1100. The retroflexion device 1010 includes an elongated actuation component 1012 which may be referenced herein, for the sake of convenience and without intent to limit, as a pull string 1012. The pull string 1012 is an elongate element having a length selected to extend along an associated flexible elongate member 1100. Furthermore, the pull string 1012 is selected to have sufficient strength to be actuated from a proximal end 1012p thereof to cause a distal end 1012d thereof to actuate the flexible elongate member 1100 with which the retroflexion device 1010 is operatively associated. The pull string 1012 may be formed of a textile, polymer, or metal monofilament, multifilament, cable-stranded element, etc., having the appropriate strength, such as selected with reference to the longitudinal extent thereof, the flexibility of the flexible elongate member 1100, etc. The flexible elongate member 1100 may be a component of a medical device, or may reference a medical device in general, such as a medical scope, reference being made simply to a flexible elongate member in a non-limiting manner, as the present disclosure need not be limited to a particular flexible elongate member or medical device to be articulated and/or retroflexed by a retroflexion device 1010 or retroflexion system 1000 formed in accordance with various principles of the present disclosure. Articulation and/or retroflexion of the flexible elongate member 1100 by the retroflexion system 1000 of the present disclosure may advantageously direct a distal working end, or the opening of a working channel (e.g., of a medical scope) to a target site within a patient's body. The working end of a medical device (either the flexible elongate member 1100, if in the form of a medical device, or a medical device extending through a working channel of a flexible elongate member in the form of a medical scope) may thereby be directed more accurately to the target site than previously achievable by prior art systems and devices.

In accordance with various principles of the present disclosure, the distal end 1012d of the pull string 1012 is operatively associated with the flexible elongate member 1100 adjacent a section of the flexible elongate member 1100 which is to be articulated by the retroflexion system 1000. For maximum efficacy, the distal end 1012d of the pull string 1012 may be positioned at and/or operatively associated with the distalmost, terminal end of the flexible elongate member 1100. However, it will be appreciated that the distal end 1012d of the pull string 1012 need not be positioned exactly at the distalmost, terminal end of the distal end 1100d of the flexible elongate member 1100, but, instead, may be adjacent, and somewhat proximal to, the distalmost, terminal end of the device to be articulated. The exact position may be determined by one of ordinary skill in the art, taking into account such factors as the flexibility of the device to be articulated, an anchored region of the pull string 1012 to allow for flexing, etc. The pull string 1012 may be operatively associated with the flexible elongate member 1100 in any of a variety of manners, the present disclosure not being limited in this regard. For example, the retroflexion system 1000 may include one or more mounts configured to operatively associate the retroflexion device 1010 with the flexible elongate member 1100. In the example of an embodiment illustrated in FIG. 1, the retroflexion system 1000 includes a distal mount 1020 having a section thereof configured to be engaged with the flexible elongate member 1100. For instance, the distal mount 1020 may include a concave section sized, shaped, configured, and/or dimensioned to receive a convex section (e.g., outer/exterior surface) of the flexible elongate member 1100. Alternatively, the distal mount 1020 may be a collar which may encircle and be affixed to (tightened, adhered, clamped, etc., with respect to) the flexible elongate member 1100. Other configurations or forms of mounts are within the scope and spirit of the present disclosure, the present disclosure not being limited in this regard.

In some aspects, the distal mount 1020 may include one or more alignment features structured, formed, shaped, positioned, or otherwise, to facilitate longitudinal and/or rotational alignment of the distal mount 1020 with respect to the flexible elongate member 1100. In some aspects, the alignment feature includes a structure with respect to which a user may be guided in rotationally orienting the system 1000 with respect to the flexible elongate member 1100. In some aspects, the structure is formed along a periphery/circumference of the distal mount 1020. In some aspects, the structure not only facilitates orientation of the distal mount 1020 with respect to the flexible elongate member 1100, but is also sized, shaped, configured, and/or dimensioned to be positioned with respect to a feature of the flexible elongate member 1100 to allow clearance for functionality of one or more features of the flexible elongate member 1100. In some embodiments, the distal mount 1020 is an end cap configured for mounting with respect to a distal end of an endoscope. In some aspects, the distal mount 1020 in the form of an end cap includes indicia for aligning with respect to an endoscope so as not to interfere with various functionalities or features of the endoscope, such as an optical system, or working channels thereof.

In the example of an embodiment illustrated in FIG. 1, the distal end 1012d of the pull string 1012 is operatively associated with the flexible elongate member 1010 via the distal mount 1020. In some aspects, the distal end 1012d of the pull string 1012 is operatively associated, such as coupled (e.g., fixedly coupled), with respect to the distal mount 1020 so that when the pull string 1012 is pulled proximally (e.g., by pulling on a proximal end 1012p thereof), the distal end 1012d pulls the distal mount 1020 proximally to flex the flexible elongate member 1100 proximally (e.g., in the direction of the illustrated flexion arrow F). In order to cause flexing of the flexible elongate member 1100 (e.g., the distal end 1100d), an actuation region 1012a of the pull string 1012 proximal to the distal end 1012d thereof is held against lateral movement (e.g., anchored) with respect to the flexible elongate member 1100 (while still being longitudinally movable along the flexible elongate member 1100). The actuation region 1012a may provide a pivot point for the pull string 1012 to flex the flexible elongate member 1100. The distance between the actuation region 1012a and the distal end 1012d of the pull string 1012 may be selected to achieve a desired bend radius. As may be appreciated by those of ordinary skill in the art, the materials of the pull string 1012 and/or the flexible elongate member 1100 may affect selection of such distance to achieve the desired articulation of the flexible elongate member 1100. It will be further appreciated that the actuation region 1012a may be positioned independently of any of the lumens/working channels through the retroflexion system 1000.

In some aspects, the actuation region 1012a of the pull string 1012 is held against lateral movement with the aid of a pivot mount to facilitate proximal pulling of the distal end 1012d thereof to cause flexing of the associated flexible elongate member 1100. More particularly, the pivot mount is mounted with respect to the flexible elongate member 1100 to provide a pivot point for the actuation region 1012a so that pulling the distal end 1012d of the pull string 1012 proximally causes the pull string 1012 to pivot about the actuation region 1012a and the pivot mount to allow the distal end 1012d of the pull string 1012 to be moved proximally by proximally flexing the flexible elongate member 1100 with which the pull string 1012 is operatively associated. In some embodiments, a pivot mount similar to the above-described distal mount 1020 may be used to limit lateral movement of the actuation region 1012a of the pull string 1012. Reference is made to the above description of a mount for the sake of brevity and without intent to limit. It will be appreciated that the axial location of the pivot mount (e.g., along the longitudinal extent of the retroflexion system 1000) may be varied to achieve the desired flexion of the distal end 1001d of the retroflexion system 1000 (and/or components thereof). In some aspects, care should be taken in selecting the location of the pivot mount with respect to the distal end 1000d of the retroflexion system 1000 (and/or flexed components thereof) so that an exposed extent of the pull string 1012 (from its attachment at the distal mount 1020 to the proximal location of the pivot mount) is limited (e.g., to reduce potential contact of the pull string 1012 with patient tissue in the region of the target site).

In some aspects, the pivot mount which holds the actuation region 1012a of the pull string 1012 with respect to the flexible elongate member 1100 may be the distal end 1030d of a flexible tubular elongate member 1030 (e.g., a sheath) extending along the flexible elongate member 1100 and defining a lumen therethrough through which the pull string 1012 translates/movably extends. The flexible tubular elongate member 1030 extends generally from a proximal end 1100p of the flexible elongate member 1100 towards a distal end 1100d of the flexible elongate member 1100. In some aspects, the flexible tubular elongate member 1030 includes a flexible elongate member 1100 engaging section facilitating engagement of the flexible tubular elongate member 1030 and the flexible elongate member 1100 to remain positioned with respect to each other, such as during movement through an anatomical passageway. In some aspects, the engaging section includes a concave recess sized, shaped, configured, and/or dimensioned to facilitate mounting of the flexible tubular elongate member 1030 with respect to the flexible elongate member 1100. For instance, the concave recess may be sized, shaped, configured, and/or dimensioned so that the flexible tubular elongate member 1030 may extend in close approximation to the exterior of the flexible elongate member 1100. For instance, the curvature of the concave recess may be selected to fit against and/or to receive the exterior of any of a variety of commercially-available endoscopes. In some aspects, the concave recess may extend more than 180° about a circumference of, and thereby partially surround the exterior of, the flexible elongate member 1100. In some aspects, the concave recess extends along a portion of the length of the flexible tubular elongate member 1030 or along substantially the entire length of the flexible tubular elongate member 1030, such as to facilitate navigation of the flexible elongate member 1100 with the system 1000 mounted with respect thereto.

In some aspects, to allow operation of the pull string 1012 and to provide an extent along which pulling of the pull string 1012 with respect to the flexible elongate member 1100 causes flexing of the flexible elongate member 1100, the flexible tubular elongate member 1030 does not extend along the entire length of the flexible elongate member 1100 (e.g., the length of the flexible tubular elongate member 1030 is less than the length of the flexible elongate member 1100). As illustrated in FIG. 1, the distalmost end 1030dm of the example of an embodiment of a flexible tubular elongate member 1030 is positioned proximal to the distal end 1100d of the flexible elongate member 1100. The pull string 1012 extends distally out of the flexible tubular elongate member 1030 and distal to the distalmost end 1030dm of the flexible tubular elongate member 1030 to be operatively associated with the distal end 1100d of the flexible elongate member 1100. As such, in some aspects, the distalmost end 1030dm of the flexible tubular elongate member 1030 may provide an anchor or pivot point for the actuation region 1012a of the pull string 1012, such as by serving as a pivot mount, and the distance between the distal end 1012d of the pull string 1012 and the actuation region 1012a may influence the bending radius of the flexed region of the flexible elongate member 1100.

As may be appreciated in view of the above, various concepts of the present disclosure may be applied to one or more devices, such as devices used in conjunction with and/or independently of the flexible elongate member 1100, such as to achieve further articulation, additional flexibility, and/or reach, optionally independently of other devices of the system. Additional devices may be extended along the exterior of the flexible elongate member 1100, and either also along the retroflexion device 1010, or through a lumen through the flexible tubular elongate member 1030 through which the pull string 1012 of the retroflexion device 1010 extends. Additionally or alternatively, additional devices may extend through a lumen defined through the flexible elongate member 1100 with which the retroflexion system 1000 is associated, such as through a working channel of a flexible elongate member 1100 in the form of a medical scope.

In some aspects, more than one actuation component, such as formed in accordance with various principles of the present disclosure as described above, may be provided. Each of the additional actuation components may operate in a manner similar to the functioning of the above-described pull string 1012. Optionally, each addition actuation component may operate in a direction different from the direction in which the above-described pull string 1012 operates. For example, if a second pull string is added to flex a distal section of one or more components of the retroflexion system 1000 in a direction opposite to the above-described flexion (such as illustrated in FIG. 1), the second pull string would have an actuation region located at a different circumferential position with respect to the distal end 1000d of the retroflexion system 1000, such as on the opposite side of (180° from) the location of the above-described actuation region 1012a of the above-described pull string 1012. The proximal ends of the one or more actuation components of the retroflexion system 1000 could be separately terminated, or they could be joined together (such as a “pulley”). As one of the actuation components is pulled proximally, the other actuation component may be unactuated (e.g., substantially slack).

When a retroflexion system 1000 formed in accordance with various principles of the present disclosure is used in conjunction with a flexible elongate member 1100 in the form of a medical scope, the retroflexion system 1000 adds further degrees of movement to a pre-existing medical scope, and thereby may enhance articulation and/or retroflexion thereof and/or of any instruments used therewith (e.g., advanced through a working channel thereof, or otherwise operatively associated with the medical scope). For instance, in some embodiments, a retroflexion system 1000 formed in accordance with various principles of the present disclosure may allow the flexible elongate member 1100 operatively associated therewith to flex greater than about 90°, such as greater than about 145°, such as greater than about 180°, and up to at least or even greater than about 210° (including various increments therebetween, such as increments of) 1°. It will be appreciated that the extent of retroflexion may, in part, be limited by the placement of the pull string 1012 and/or other components of the retroflexion system 1000 with respect to the flexible elongate member 1100 which may interfere with relative movement and positioning of the flexed portion of the retroflexion system 1000 and flexible elongate member 1100 with respect to proximal portions thereof. In some aspects, the pull string 1012 may be tuned to work in conjunction with the retroflexion system 1000 such that full maneuverability of the retroflexion system 1000 is restored, while not over-extending, hyperflexing the retroflexion system 1000 in ways not designed by the manufacturer of the retroflexion system 1000.

As may be appreciated, as the distal portions 1000d, 1100d of the retroflexion system 1000 and flexible elongate member 1100 are retroflexed, such distal portions 1000d, 1100d approach the portions of the retroflexion system 1000 and flexible elongate member 1100 proximal to the bent portions thereof. However, various properties of the retroflexion system 1000 and flexible elongate member 1100 (or at least the distal portions 1000d, 1100d thereof), such as stiffness, resiliency, bendability, etc., typically will create a bending radius which spaces the distal portions 1000d, 1100d of the retroflexion system 1000 and flexible elongate member 1100 from the portions proximal thereto as the retroflexion system 1000 and flexible elongate member 1100 are retroflexed. Such spacing typically allows the distal portions 1000d, 1100d to be retroflexed greater than 180° since the spacing typically prevents the portions of the retroflexion system 1000 and flexible elongate member 1100 which are proximal to the distal portions 1000d, 1100d from physically interfering with retroflexion. In some aspects, the distal portions 1000d, 1100d of the retroflexion system 1000 and flexible elongate member 1100 are capable of being retroflexed greater than 180°, and even as much as 210° before contacting the portions of the retroflexion system 1000 and flexible elongate member 1100 proximal thereto. Such retroflexion is greater than previously achievable by existing devices and allows the flexible elongate member 1100 to access a greater range of target sites. It will be appreciated that the closer the distal portions 1000d, 1100d of the retroflexion system 1000 and flexible elongate member 1100 are bent toward the proximal portions of the retroflexion system 1000 and flexible elongate member 1100, the more distalmost ends of the retroflexion system 1000 and flexible elongate member 1100 face the proximal portions, and the more the proximal portions may interfere with use of the distal portions 1000d, 1100d.

In some aspects, such retroflexion allows access to target sites which are not along the anatomical passageway through which the flexible elongate member 1100 is inserted (e.g., not distal or otherwise longitudinally along the insertion path of the flexible elongate member 1100). More particularly, if the flexible elongate member 1100 is inserted through an anatomical passageway or structure in a first direction, and the target site is not aligned with that first direction (e.g., is positioned 45° or 90° or more from a path or direction of insertion or advancement of the flexible elongate member 1100), the distal end 1100d of the flexible elongate member 1100 must be flexed to access the target site, but various constraints on prior systems and devices have interfered with achieving the necessary degree of flexion. For instance, medical professionals have faced challenges accessing the fundus of a patient's stomach with an endoscope inserted through a patient's mouth and esophagus because of the lateral position of the fundus with respect to the lower esophageal sphincter. In some aspects, the patient's esophagus does not afford the flexible elongate member 1100 significant lateral movement for access to target sites such as the fundus. A system 1000 and retroflexion device 1010 formed in accordance with various principles of the present disclosure allow access to target sites which were previously difficult to access, such as the fundus of a patient's stomach. Various procedures may thus be performed, such as GERD repair in the region of the fundus, which had been difficult if not impossible to be performed endoscopically, transluminally, etc. For instance, a retroflexion system 1000 formed in accordance with various principles of the present disclosure allows sufficient retroflexion such that when advanced through a patient's esophagus into the patient's stomach, the distal end 1000d of the retroflexed component of the system 1000 may be sufficiently retroflexed into a direction generally aligned with the cardia/fundus of the stomach. Procedures in such regions of the stomach which had previously been difficult to perform, such as GERD repair, may be performed with the use of a retroflexed component of the retroflexion system 1000 of the present disclosure.

In embodiments of a retroflexion system 1000 in which the pull string 1012 extends along/through a flexible tubular elongate member 1030, the flexible tubular elongate member 1030 may optionally include additional longitudinally-extending lumens therethrough. For instance, in some embodiments, the flexible tubular elongate member 1030 may define auxiliary working channels to supplement one or more lumens/working channels through the flexible elongate member 1100 upon which the retroflexion system 1000 operates. Such auxiliary working channels may allow the use of additional devices with the flexible elongate member 1100 and/or the retroflexion system 1000, optionally in conjunction with the retroflexion device 1010 of the present disclosure. In some aspects, a retroflexion device 1010 formed in accordance with various principles of the present disclosure not only articulates a flexible elongate member 1100 (e.g., the distal end thereof), such as to direct a device at or extending from the distal end 1100d thereof, but also may facilitate navigation of additional devices used in conjunction with the flexible elongate member 1100 and/or the retroflexion system 1000. In some aspects, the advancement, retraction, insertion, etc., of additional devices with a flexible elongate member 1100 may add bulk to the flexible elongate member 1100 and/or the overall system. The retroflexion system 1000 and device 1100 of the present disclosure provides an independent flexion system and is designed to address such challenges and to overcome the resistance to flexion which may be increased by the use of additional devices with the flexible elongate member 1100.

In some aspects, it may be desirable to achieve greater articulation of devices used in conjunction with a flexible elongate member 1100 (e.g., a medical scope) than achievable by the flexible elongate member 1100 on its own (e.g., articulation with the use of an articulation system provided with a medical scope, even if the scope is brand new). In some aspects, a retroflexion device 1010 or retroflexion system 1000 formed in accordance with various principles of the present disclosure may be operatively associated individually/separately with other flexible elongate members, such as elongate components of other tools, instruments, etc., such as used in conjunction with a flexible elongate member 1100 and/or retroflexion system 1000 as described herein. A retroflexion device 1010 and system 1000 formed in accordance with various principles of the present disclosure provides greater articulation than previously achievable, such as by existing articulation systems of existing medical scopes.

It will be appreciated that reference to pulling on the pull string 1012 proximally to actuate the actuation region 1012a of the pull string 1012 need not be in precisely a proximal direction with respect to the retroflexion system 1000. Instead, such proximal pulling may more broadly reference proximal pulling on the pull string 1012 to cause the portion of the pull string 1012 which extends along the flexible elongate member 1200 to move proximally. The specific direction in which the proximal end 1012p of the pull string 1012 is pulled may be affected by the manner in which the proximal end 1012p of the pull string 1012 is operatively associated with the proximal end 1030p of the flexible tubular elongate member 1030. As noted above, a pull string 1012 of a retroflexion system 1000 formed in accordance with various principles of the present disclosure is accessible along (at or adjacent) the proximal end 1012p thereof. For instance, the pull string 1012 extends from an actuation section along a distal end 1012d thereof (and along the distal end 1100d of the flexible elongate member 1100 which the pull string 1012 actuates) to be accessible along a proximal end 1012p thereof.

In the example of an embodiment illustrated in FIG. 1, the proximal end 1012p of the pull string 1012 is operatively associated with an adaptor/proximal mount 1022 provided along a proximal end 1000p of the retroflexion system 1000, such as along a proximal end 1030p of a flexible tubular elongate member 1030 along which the pull string 1012 of the retroflexion device 1010 extends. In some aspects, the proximal mount 1022 is configured to facilitate advancement of devices through lumens defined within the flexible tubular elongate member 1030, such as by providing ports 1023, 1025 facilitating access to the lumens defined through the flexible tubular elongate member 1030. In some aspects, the proximal mount 1022 is configured to be operatively associated with a proximal end of the flexible elongate member 1100. For instance, a bracket or other mounting device may be provided to mount the proximal mount 1022 with respect to a proximal end of the flexible elongate member 1100, such as with respect to a handle of the flexible elongate member 1100.

In some aspects, the proximal end 1012p of the pull string 1012 is extended along the flexible elongate member 1100 and through the proximal mount 1022. In such manner, the proximal mount 1022 may support a portion of the pull string 1012 along the proximal end 1012p thereof, such as with respect to the flexible elongate member 1100. Positioning of the proximal end 1012p of the pull string 1012 at or along the proximal mount 1022 may facilitate control of both the flexible tubular elongate member 1030 and any devices associated therewith (e.g., extending therethrough or therealong) as well as control of the pull string 1012 at a similar location along the retroflexion system 1000.

Depending on the position, orientation, etc., of the proximal mount 1022 with respect to the flexible elongate member 1100, and the association of the pull string 1012 with the proximal mount 1022, the proximal end 1102p of the pull string 1102 may be pulled axially or transverse with respect to the longitudinal extent of the flexible elongate member 1100 and the section of the pull string 1012 extending therealong to the distal end 1010d of the retroflexion device 1010. In the example of an embodiment illustrated in FIG. 1, the pull string 1012 extends through the proximal mount 1022 such that the proximal end 1012p of the pull string 1012 extends transversely out of the proximal mount 1022 with respect to the longitudinal extent of the remaining portion of the pull string 1012 (extending distally to the distal end 1010d of the retroflexion device 1010). As such, pulling the proximal end 1012p of the pull string 1012 generally transverse to the longitudinal extent of the retroflexion system 1000 (e.g., in the direction of pull arrow P) causes proximal movement of the pull string 1012 to cause retroflexion of the flexible elongate member 1100 with respect to which the retroflexion device 1010 is operatively associated.

In the example of an embodiment illustrated in FIG. 1, the pull string 1012 includes a user engagement component, such as a pull handle 1014, at a proximal end 1012p thereof. The pull handle 1014 is sized, shaped, configured, and/or dimensioned to facilitate engagement of the proximal end 1012p of the pull string 1012 by a user. In some aspects, the pull handle 1104 is configured to enable a firm grip as well as sufficient pull force on the pull string 1102 to effect the desired retroflexion. For instance, the pull handle 1104 may extend generally transverse to the longitudinal extent of the pull string 1102.

In some aspects, the retroflexion system 1000 includes a mount 1024 for the pull handle 1014 of the pull string 1012. The pull handle mount 1024 is sized, shaped, configured, and/or dimensioned to engage the pull handle 1014 to hold the pull handle 1014 in place and not be moved so that the flexible elongate member 1100 is not flexed. For instance, the pull handle mount 1024 may engage the pull handle 1014 via an interference or friction fit. Optionally, the pull handle mount 1024 is provided on the proximal mount 1022 of the retroflexion system 1000. As such, the pull handle 1014 remains available in generally the same location as other devices which may be manipulated with respect to the flexible elongate member 1100 and/or the flexible tubular elongate member 1030 at the proximal end 1000p of the retroflexion system 1000. The pull handle 1014 can be engaged with the pull handle mount 1024 while other features of the retroflexion system 1000 are being used.

In some embodiments, the retroflexion system 1000 includes a locking system including a pull string lock 1026 configured to operatively engage a portion of the pull string 1012, such as to secure the pull string 1012 in a tensioned state. An example of an embodiment of a pull string lock 1026 is illustrated in FIG. 1, and in the detail view of FIG. 1A, in the form of a post or other elongated element about which the pull string 1012 may be wrapped or otherwise secured. However, it will be appreciated that the present disclosure need not be limited by the configuration of the pull string lock 1026. When the pull string 1012 is pulled proximally and a desired degree of retroflexion of the flexible elongate member 1100 is achieved, the tension on the pull string 1012 may be temporarily maintained (and the retroflex configuration fixed) by wrapping the proximal end 1012p of the pull string 1012 around the pull string lock 1026. In some embodiments, the pull string lock 1026 includes a locking structure 1028, such as an element which assists in retaining the pull string 1012 with respect to (e.g., wrapped tightly around) the pull string lock 1026. In some embodiments, the locking structure 1028 is formed to provide an interference fit or frictional engagement of the pull string 1012 with respect to the pull string lock 1026. For instance, the locking structure 1028 may be an element which frictionally engages the pull string 1012, such as a resilient rubber washer secured over the pull string lock 1026 (and optionally on a seat formed on the pull string lock 1026), such as illustrated in greater detail in the detail view provided in FIG. 1A. Once the desired retroflexion of the flexible elongate member 1100 is attained, the pull string 1012 tension may be retained by wrapping the proximal end 1012p of the pull string 1012 around the pull string lock 1026, between the locking structure 1028 and a seat 1027 on the pull string lock 1026 between the locking structure 1028 and the structure on which the pull string lock 1026 is provided. In some aspects, compression of the pull string 1012 between the locking structure 1028 and the seat 1027 retains the pull string 1012 in position with respect to the pull string lock 1026.

In the illustrated example of an embodiment, the pull string lock 1026 is provided on/extends from the proximal mount 1022 of the retroflexion system 1000. In some aspects, the pull string lock 1026 may be sufficiently close to the pull handle mount 1024 to facilitate convenient mounting of the pull handle 1014 on the pull handle mount 1024 either after release from the pull string lock 1026, or optionally even when the proximal end 1012p of the pull string 1012 is wrapped around the pull string lock 1026.

In view of the above, it may be appreciated that a retroflexion system 1000 formed in accordance with various principles of the present disclosure may be used in conjunction with an articulation mechanism of the flexible elongate member 1100 with which the retroflexion system 1000 is operated. In such manner, the retroflexion system 1000 operates as an assist to the existing system of the flexible elongate member 1100, such as to normalize the articulation functionality of the pre-existing system of the flexible elongate member 1100. The retroflexion system 1000 of the present disclosure ensures that the desired degree of retroflexion is achieved for the procedure to be performed with the use of the flexible elongate member 1100. In some aspects, a retroflexion system 1000 formed in accordance with various principles of the present disclosure to be used in conjunction with an existing device or system, and mounted with respect to such device or system, facilitates articulation. For instance, pulling of an actuation device, such as the pull string 1012 of the above-described example of an embodiment of a retroflexion system 1000, extending along/alongside the exterior/outside of the device or system to be actuated applies a tangential force which may apply a greater force to the device or system than a force applied by an actuation element extending therethrough (within, rather than outside, the device/system).

Various principles of the present disclosure such as those described above may be implemented in a variety of manners and/or applied to a variety of medical devices, examples of which will now be described. It will be appreciated that various methods described with respect to the described examples of embodiments may be applied to other embodiments, such as described more broadly/generically above.

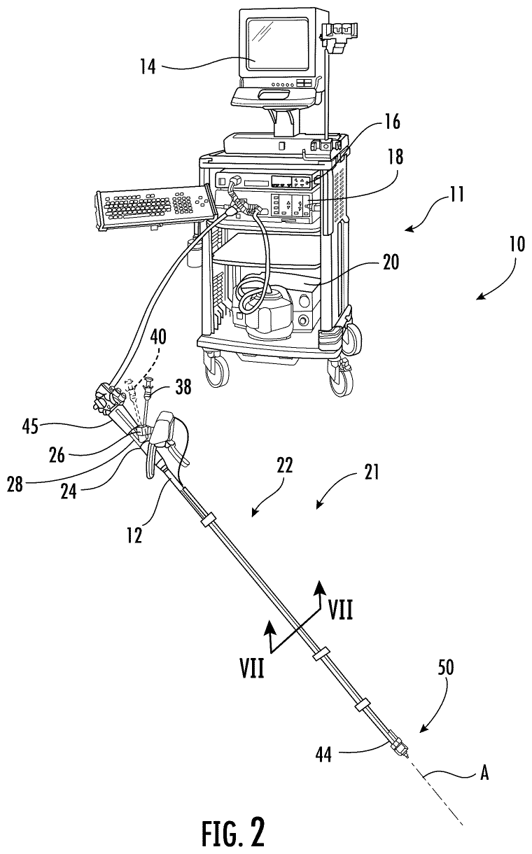

Referring to FIG. 2 through FIG. 5, an endoscopic treatment system 10 includes an endoscope system 11 and an endoscopic suturing system 21. The endoscope system 11 includes an endoscope 12, a video display unit 14, an image processing device 16, a light source 18, and a suction device 20. In accordance with an embodiment, the endoscope 12 has a small profile, generally 5-10 mm in diameter. However, the size of the endoscope is not critical, and elements described herein can be adapted for endoscopes of other sizes. In the example of an embodiment shown, the endoscope 12 has a single instrument channel 13 and an optical lens 15 (FIG. 4). However, the endoscope 12 may have more than one instrument channel or no instrument channel all, as operation of the system does not necessarily require use of the instrument channel through the endoscope. The endoscope 12 includes a distal end 44 and a proximal end 45 and a longitudinal axis A extending therebetween.

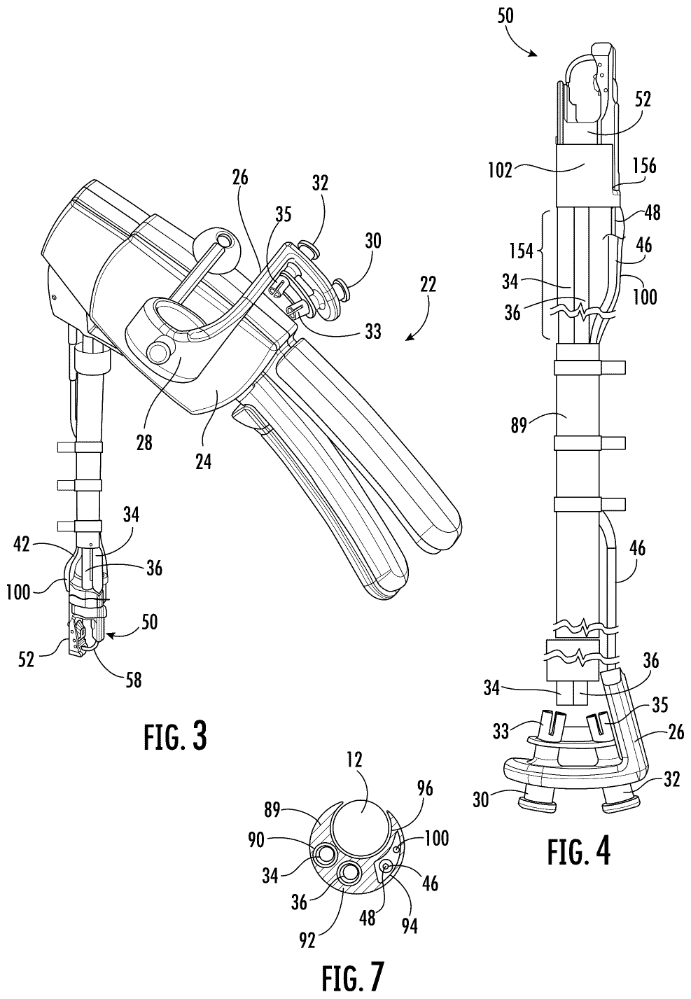

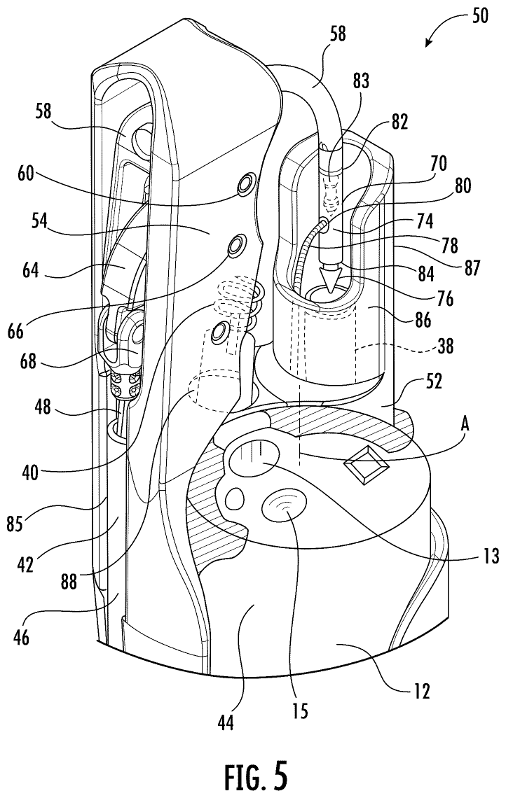

The suturing system 21 includes a suturing device 22 (FIG. 3), a needle assembly 70 (FIG. 5) movable through tissue by the suturing device 22, and first and second devices 38, 40 used in association with the suturing device 22 (FIG. 5).

Referring to FIGS. 2, 3, and 4, the suturing device 22 has a proximal operable handle 24 provided with a mounting bracket 26 and a collar 28 at which the handle is removably coupled to endoscope 12. The bracket 26 includes first and second instrument ports 30, 32 at which instruments can be received into first and second catheters 34, 36, respectively. First and second tubular connectors 33, 35 are aligned with the ports 30, 32 that couple the ports 30, 32 to the first and second catheters 34, 36.

Referring to FIG. 3, FIG. 4, and FIG. 7, a transmission assembly 42 includes a transmission sheath 46 and a transmission cable 48 displaceable within the transmission sheath 46, both coupled relative to the handle 24. The transmission sheath 46 is coupled relative to a first portion of the handle (i.e., a stationary member), and the transmission cable 48 is coupled to a second portion of the handle (i.e., a movable lever), such that when the handle 24 is operated the cable 48 is displaced within the transmission sheath 46.

The first and second catheters 34, 36 and the transmission assembly 42 extend from the proximal handle 24, along the outside of the endoscope 12, to a distal cap assembly 50. The distal cap assembly 50 is adapted to be mounted at the distal end 44 of the endoscope 12, and the handle 24 remotely operates the cap assembly 50 via the transmission assembly 42.

Referring to FIG. 5, the cap assembly 50 includes a mount 52, U-shaped support bracket 54 extending distally from the mount, and a needle arm 58 rotatably mounted on the bracket 54 with a first pin 60. A bell crank 64 is rotatably mounted at a second pin 66 on the support bracket 54 and engages the needle arm 58 at intermeshing gears (not shown). The distal end of the transmission cable 48 of the transmission assembly 42 is attached to the bell crank 64 at a clevis 68. When the transmission assembly 42 is operated by the handle 24, it results in rotation of the bell crank 64 and consequent rotation of the needle arm 58 between the open and closed positions.

The needle assembly 70 is coupled to a needle mount 83 at an end of the needle arm 58. The needle assembly 70 includes a tubular needle body 74, a needle tip 76, and suture 78 coupled to the needle body. The needle body 74 includes a side opening 80 through which the suture 78 extends, a first end 82 at which the needle assembly is coupled to the needle mount 83, and a second end 84 to which the tip 76 is coupled. The tip 76 defines a tissue-piercing taper. The suture 78 may be formed of any materials commonly available for surgical suture such as nylon, polyolefins, PLA, PGA, stainless steel, nitinol, and others. One suitable needle assembly is described in more detail in U.S. Pat. No. 9,198,562, which patent is hereby incorporated herein by reference in its entirety for all purposes.

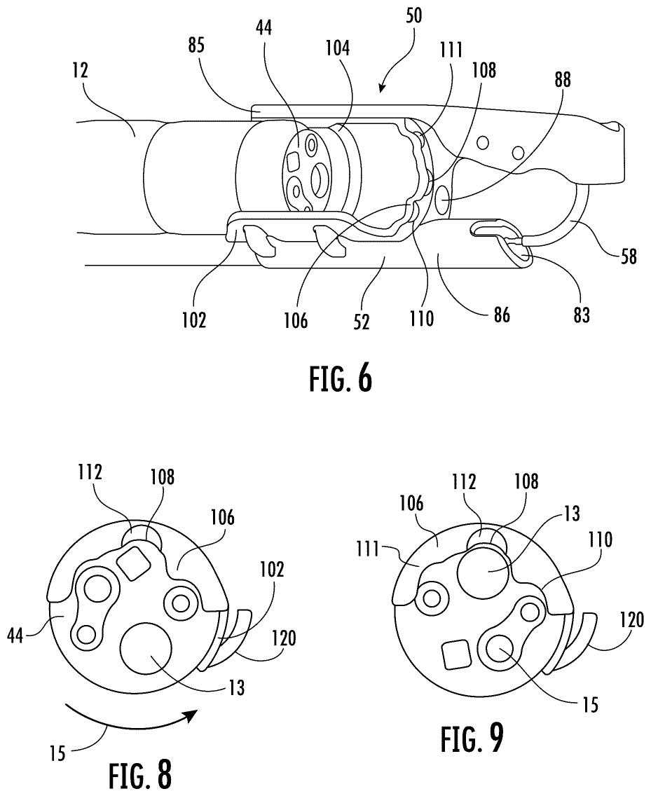

Referring to FIG. 5 and FIG. 6, the mount 52 of the cap assembly 50 includes a side recess 85 into which the transmission assembly 42 is received, and a first throughbore 86 and a second throughbore 88. The first throughbore 86 is positioned in alignment with both the needle mount 83 of the needle arm 58 and needle assembly 70 when the needle arm 58 is in the closed position. A tissue guide 87 extends distally on the mount 52 from over the first throughbore 86 and provides a surface on which to stabilize tissue as it is pierced by the needle assembly 70. The second throughbore 88 is positioned between the first throughbore 86 and the support bracket 54. More particularly, the axial center of the second throughbore 88 is positioned between the first throughbore 86 and the pin 60 (or axis) on which the end effector and/or assembly 58 rotates. The first and second throughbores 86, 88 may be parallel to each other and the longitudinal axis A of the endoscope, or the second throughbore 88 may be obliquely angled relative to the first throughbore 86 so as to direct the second device 40 at a particular orientation into the needle path, as described further below. The mount 52 is structured such that when the cap assembly 50 is coupled to the endoscope 12, as described below, the first and second throughbores 86, 88 are positioned radially outside the profile of the endoscope 12.

Referring to FIG. 4 and FIG. 7, the distal end of the first catheter 34 is fixed in the first throughbore 86, and its proximal end is coupled to a first connector 33 on the handle bracket 26. The distal end of the second catheter 36 is fixed in the second throughbore 88, and its proximal end is coupled the second connector 35 on the handle bracket 26. The first and second catheters 34, 36 may be discrete catheters or may be separate lumens of a common catheter. In a preferred embodiment, the catheters 34, 36 are discrete catheters covered in a preferably crescent moon shaped common sheath 89 along substantially all (i.e., at least 80 percent, and more preferably at least 90 percent) of their length. Alternatively, the common sheath 89 may extend along the entire length of the catheters 34, 36, a partial length thereof, or may be provided in sections along selected portions of the catheters 34, 36. In a preferred embodiment, the common sheath 89 is flexible, defines a lumen 90 for catheter 34, a lumen 92 for catheter 36, and a lumen 94 for both transmission assembly 42 and a retroflex pull string 100, the function of which is described below. Optionally, separate lumens can be provided for the transmission assembly 42 and the pull string 100. A concave recess 96 is provided along the length of the sheath 89 so that the sheath is preferably adapted to extend in close approximation to the exterior of an appropriately sized endoscope 12. In an embodiment, the sheath 89 is adapted to accommodate the endoscope such that the sheath preferably extends more than 180° about a circumference of, and thereby partially surrounds the exterior, of the appropriately sized endoscope 12.

Referring to FIG. 4 and FIG. 5, the first catheter 34 is adapted to receive a first device 38 that has a distal end effector that can receive and grasp the needle assembly 70. The second catheter 36 is adapted to receive a second device 40 that has a distal end effector that can engage tissue, and draw the tissue back into the path of the needle so that the tissue can be pierced by the needle assembly 70 as the needle assembly 70 is moved from the open to the closed position.

Referring to FIG. 6, the cap assembly 50 is secured to the distal end 44 of the endoscope 12 with a peripheral engagement structure that is adapted to be positioned about approximately 180°, and preferably slightly greater than 180°, around the circumference of the distal end of the endoscope 12. In one embodiment, the structure is a resilient clip 102 integrated at the proximal end of the mount 52. The clip 102 includes an opening 104 to allow the distal end 44 of the endoscope 12 access therethrough. The clip 102 may be formed from ABS plastic, other suitable plastics, elastic materials, as well as polymer-coated metals.

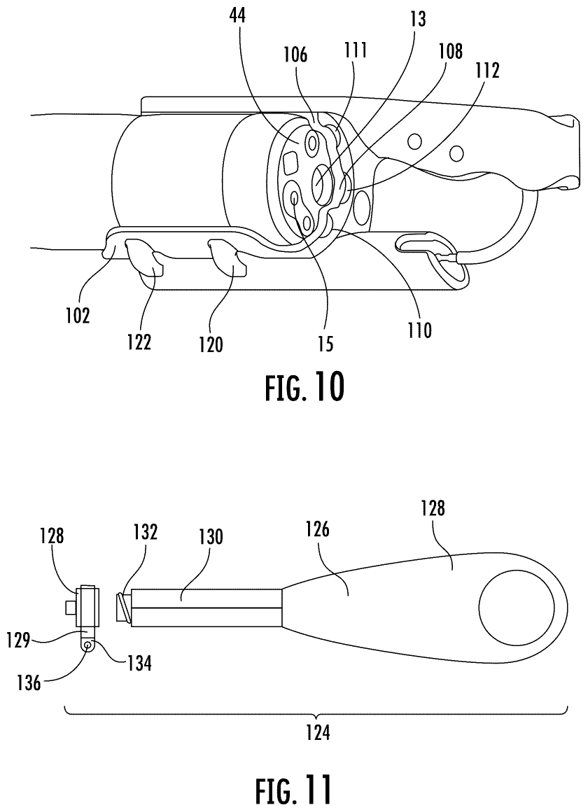

Turning to FIG. 6, FIG. 8, FIG. 9, and FIG. 10, a stop 106 is formed at the distal end of the clip 102. The distal end 44 of the endoscope 12 is adapted to pushed into the recess 104 of the clip 102 and seat against the stop 106. The stop 106 preferably includes a largest first clearance window, formed as a first recess 108 in the circumference of the stop 106 to permit pass through access to instrument channel 13 on various endoscopes of multiple manufacturers when the endoscope is properly rotationally aligned in the clip 102, and smaller second and third clearance windows, formed as second and third recesses 110, 111 approximately 60° on either side of the first recess 108, to prevent obstruction of the optical lens 15 and other working features of various endoscopes. The first recess 108 is located radially inward of the instrument channel lumen 88. In order to align the features of the endoscope relative to such first, second and third recesses 108, 110, 111, the stop 106 includes an indicia 112 relative to which the instrument channel 13 or other designated feature of an endoscope is rotationally aligned. For example, if an endoscope does not include an instrument channel 13 (as such is not required with the system described herein), the optical lens could be designated to be aligned with the indicia 112. In a preferred embodiment, the indicia 112 is a contrasting color relative to a color of a majority of the cap assembly which is provided about the first recess 108. With such rotational alignment of the designated endoscope feature relative to the indicia 112 (in the direction of arrow 115), the optical lens 15 of the endoscope is assured unobstructed view and appropriate orientation relative to the suturing arm 58, instrument channel lumen 88, and needle exchange lumen 86, regardless of the major manufacturer endoscope utilized with the system, without necessitating activating the endoscope. That is, the relative orientation, access, and field of view can be ascertain without requiring powering up the endoscope and previewing the field of view from the lens 13 on the display 14 in advance of the procedure. This significantly reduces preparation time prior to the procedure. The location of the first and second recesses 108, 110 and the indicia 112 relative to the suturing arm 58, instrument channel lumen 88, and needle exchange lumen 86, as well as areas which will be covered and blocked from view at the distal end of the endoscope, is based on analyses of scans of multiple distal ends of endoscopes.

Referring to FIG. 10 the resilient clip 102 further includes a plurality of longitudinally displaced (proximal and distal) integrated hooks 120, 122. In an embodiment, exactly two hooks are provided to the clip. The hooks are preferably identified with contrasting color relative to surrounding areas of the end cap and to a majority of the cap assembly. The hooks 120, 122 may also be numbered with indicia, e.g. ‘1’ and ‘2’, according to the intended order of use, as further described below.

Turning to FIG. 11, the system also includes a tape applicator 124 for applying tape at each of the hooks 120, 122. The tape applicator 124 includes a handle 126 and a spool 128 of tape 129 on a distal end of the handle. The illustrated example of an embodiment of a handle 126 is preferably monolithic, including a wider proximal end 126p for stable gripping in a user's hand, and a narrower extension 130 for unobtrusively reaching about the distal end 44 of the endoscope 12 and cap assembly 50 during tape application. The extension 130 includes a spool mount 132. In a preferred embodiment the spool mount 132 is in the form of an external thread. The spool 128 is rotatably fixed on the spool mount 132 of the handle 126 during use. The spool 128 includes an internal thread (not shown) that allows the spool to be rotated onto the spool mount 132 in a rotational direction opposite the intended application of the tape, such that when tension is applied to the tape the spool is pulled in a direction that rotationally secures the spool relative to the mount. Other securing interfaces between the mount and spool can be used. The tape preferably has a non-adhesive free end 134 provided with an integrated loop 136 that is reinforced and sized for attachment to one of the hooks 120, 122. The reinforced integrated loop 136 is preferably provided in a color to facilitate locating it in the environment of an operating room. By way of example, the loop 136 is preferably opaque; and the loop preferably matches color with the hooks on the cap assembly. Both may be opaque white. In an embodiment, the loop 136 has a diameter of approximately 2.5 mm. The diameter of the spool 128 is preferably less than the diameter of the endoscope 12 to provide mechanical advantage when wrapping the tape 129 to secure the clip 102 and endoscope 12 relative to each other. In one embodiment, the spool has a diameter of 10 mm and a tape width of 5 mm. In an embodiment, except for the free end 134, the tape 129 has a consistent adhesion along its length and is primarily adapted to stick to the back of itself under tension, as well as both the endoscope 12 and the clip 102. Alternatively, the tape 129 can have a non-stick first portion for first wrapping about the endoscope, and a second adhesive portion for wrapping over and adhering to the back of the first portion and the back of the second portion.

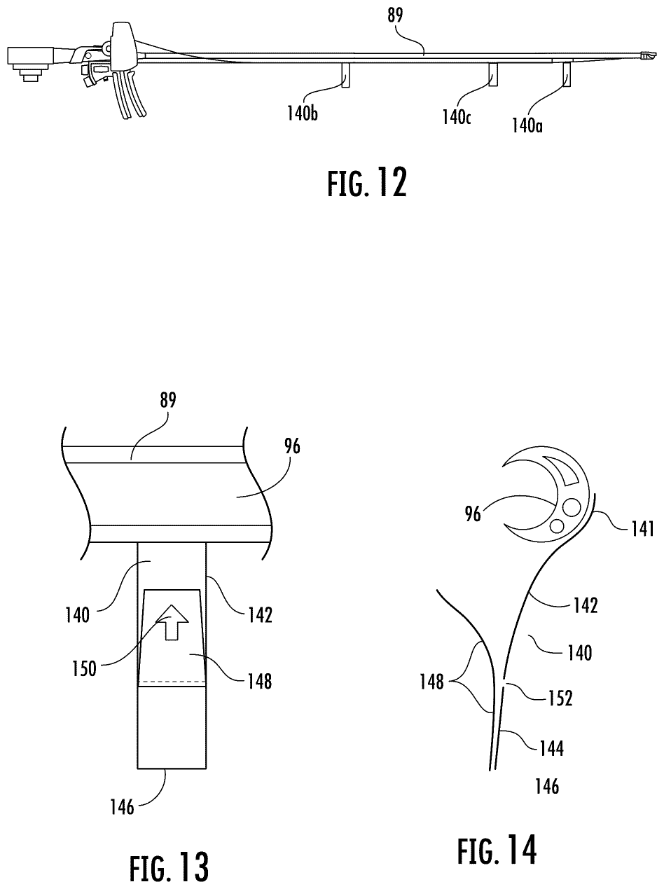

Turning now to FIG. 12, FIG. 13, and FIG. 14, a tape system is also provided for securing a portion of the sheath relative to the endoscope. The sheath 89 is provided with a plurality of strips of tape 140 pre-disposed along the length of the sheath 89. In a preferred embodiment, three strips of tape are used; a first strip 140a located at a distal end of the sheath, a second strip 140b located at a substantially central location along the length of the sheath, and a third strip 140c located between the first and second strips 140a, 140b and preferably closer to the first strip 140a. This limited use of tape and particular spacing has been shown to provide good stabilization of the sheath relative to the endoscope during use. That said, additional strips of tape can be pre-provided to the sheath and used in the manner described. In accordance with one aspect of the system, the strips of tape 140 have an adhered first portion 141 pre-applied to the sheath 89, a second portion 142 close to the sheath recess 96 that is non-sticky to prevent adhesion to the endoscope as the tape is wrapped around the endoscope, and a third portion 144 extending from the second portion 142 and to a free end 146 of the tape strip that is adhesive and adapted to adhere to the back of the tape when it is wrapped around the endoscope 12 and sheath 89. In accordance with another aspect, the tape strips 140 each include a pull off protective strip 148 that covers the second portion 144. The pull off protective strip 148 extends to cover the third portion 144 of the tape and is bent into an L-shaped extending tab between the free end 146 and the first portion 141. The tab 148 is preferably a high visibility color, contrasting with the sheath, e.g., white, and also preferably includes indicia, e.g., a laser-cut arrow 150, indicating the direction in which the tab 148 of the protective strip should be pulled to release the protective strip from the third portion to expose the adhesive (FIG. 13). In accordance with one aspect of the system, the strips 140 have at least a perforation 152 that permits breakaway disassembly after use of the system (FIG. 14). The perforation 152 is preferably provided at or near the junction of the second and third portions 142, 144 of the tape.

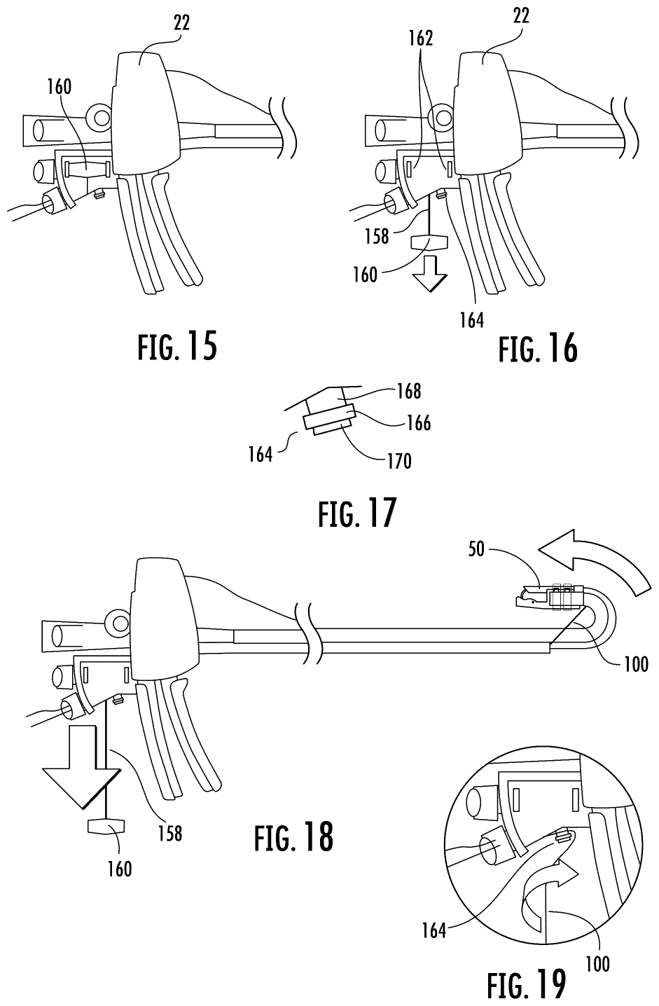

Turning to FIG. 4, FIG. 15, and FIG. 16, in accordance with another aspect of the system, the cap assembly 50 is adapted to retroflex relative to the sheath 89 via actuation from the proximal handle 24. The pull string 100, referenced above, extends from the proximal handle 24, through lumen 94 in the sheath 89, and exits the distal end of the sheath. A pull string 100 herein refers to a textile, polymer, or metal monofilament, multifilament, or cable stranded element. Between the distal end of the sheath and the end cap there is gap 154 (FIG. 4) exposing a length of the catheters 34, 36, the transmission assembly 42, and the pull string 100 outside the sheath 89 and to the cap assembly 50. The length of the gap 154 generally represents the difference in length between the endoscope 12, which has a longer first length, and the sheath 89, which has a shorter second length. In a preferred embodiment the exposed length of the gap 154 is less than 16 cm; more preferably the length of the gap is between 6 and 12 cm; even more preferably the length of the gap is between 7.5 and 10 cm; and most preferably the exposed length of the gap is 8.5 cm to provide optimal retroflexion, as described below. On the opposite side of the gap 154, the transmission assembly 42 is secured in the side recess 85 of the cap assembly 50 and the catheters 34, 36 are secured within the first and second throughbores 86, 88 (e.g., as illustrated in FIG. 5). Further, a distal end 156 of the pull string 100 is secured to the cap assembly 50. In one assembly, the distal end 156 is bonded into a hole (not shown) at the proximal end of the cap assembly 50. The proximal end 158 of the pull string 100 exits from the sheath and through an opening in the proximal handle 24 and is attached to a pull handle 160. A pull handle mount 162 is preferably formed on the proximal handle 24 and can secure the pull handle 160 via interference fit when not in use. A pull string lock 164 is provided on the proximal handle 24 to secure the pull string in a tensioned state. An example of an embodiment of a pull string lock 164 includes a resilient rubber washer 166 secured on a seat 168 and over a post 170 (FIG. 17). In use, when it is desirable to retroflex the cap assembly 50, the pull handle 160 can be released from the handle mount 162 and pulled to apply tension to the pull string 100. As tension is applied to the pull string 100, the pull string 100 is retracted across the gap 154 and through the lumen 94. As the pull string 100 is pulled across the gap 154, the cap assembly 150 is retroflexed to shorten the distance between the end of the sheath 89 and the location at which the distal end 156 of the string is attached to the cap assembly 50, as shown in FIG. 18. When a desired degree of retroflex is obtained, the tension on the pull string 100 may be temporarily maintained (and the retroflex configuration fixed) by wrapping the proximal end 158 of the pull string 100 around the post 170 between the washer 166 and the seat 168 (FIG. 19). Compression of the pull string 100 between the washer 166 and the seat 168 retains the pull string in position. The pull handle 160 can be placed back on the handle mount 162 to keep it out of the way (i.e., back to the position shown in FIG. 15) while other features of the proximal handle 24 and the endoscope 12 are used. Retroflex of the system may be used in combination with actuating retroflex of the endoscope. Then, when it is intended to release the retroflex condition at the distal end, the proximal end 158 of the pull string 100 is unwound from the pull string lock 164, and tension on the pull string is released. In such manner, retroflex of the system operates as an assist to the system attached to the endoscope and ensures that retroflex of the endoscope is carried out full in the manner intended. It is also recognized that this type of retroflex system, with or without assist from the endoscope, can be used in different endoscopic therapeutic and interventional systems, and is not limited to systems for suturing.

In light of the above, the suturing device may be prepared for use in conjunction with an endoscope as follows. The distal end 44 of the endoscope 12 is pushed into the resilient clip 104 until seated at or near the distal stop 106 (FIG. 6). Then, the endoscope and cap assembly 50 are coaxially rotated relative to each other until the instrument channel 13 of the endoscope is rotationally aligned with the indicia 112 at the distal stop 106 to ensure appropriate alignment between the two (FIG. 8 and FIG. 9). Then, if necessary, the endoscope 12 is pushed all the way into contact with the distal stop 106 (FIG. 10).

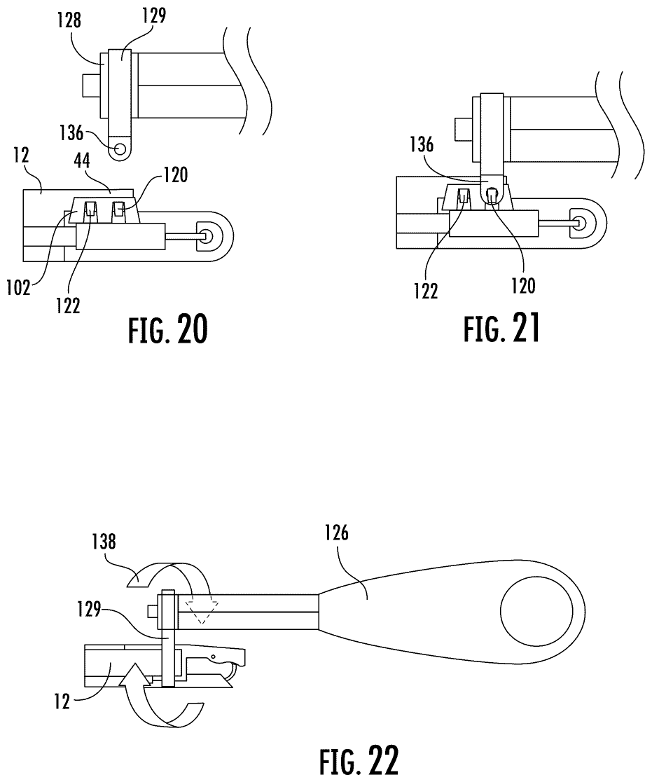

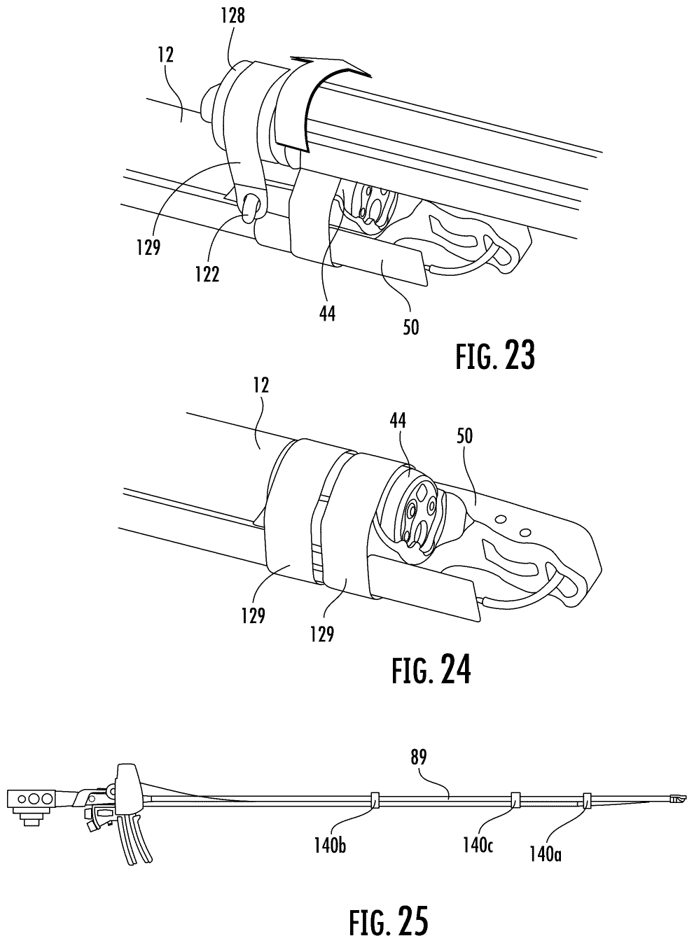

Then, referring to FIGS. 19, 20 and 21, the loop 136 at the free end 134 of a first spool 128 of a tape applicator 124 is engaged over the distal first hook 120 and the handle 126 coupled to the spool 128 is maneuvered to wrap the tape 129 over a distalmost portion of the distal end 44 of the endoscope 12, over the clip 102, and back and around over the back of the tape 129 in a circular motion (arrow 138) one or more times to secure the endoscope relative to the clip. While wrapping, tension is applied to the tape. The relative diameters of the tape spool 128 and the endoscope 12 facilitate application of significant tension on the tape during wrapping of the endoscope with the tape. After the tape is applied at the first hook 120, the depleted spool 128 is removed from the spool mount 132, and a fresh spool 128 of tape is attached at the spool mount of the handle 126. Referring to FIG. 23 and FIG. 24 the tape 129 from the fresh spool 128 is similarly applied starting from the second hook 122 and over an adjacent area of the endoscope 12 until the distal end 44 of the endoscope 12 and cap assembly 50 are secured together.

Then, turning to FIG. 7, FIG. 13, FIG. 14, and FIG. 25, the endoscope is fully pressed into the recess 96 along the length of the sheath 89. Starting from the distal end of the system, and at strip of tape 140a, the tab 148 of the protective strip is pulled off to expose the adhesive portion 144, the tape 140 is pulled taut, the first portion 142 of the tape is wrapped around the endoscope 12, and the second adhesive portion 144 of the tape is adhered to the sheath on the opposite side of the recess 96 and then extended over the tape again to secure the endoscope thereat. The process is repeated along the length of the sheath for remaining strips 140c, then 140b, to properly secure the sheath relative to the endoscope. This longitudinally secures the sheath 89 to the exterior of the endoscope 12 for in vivo operation.

The collar 28 is properly positioned at the proximal handle 45 of the endoscope 12. The first device, a needle capture instrument 38 loaded with a needle assembly 70, is advanced through the first port 32, into the first lumen 34 and to the cap assembly 50. Suitable needle capture devices 38 are described in detail in U.S. Pat. No. 8,679,136, which patent is hereby incorporated herein by reference in its entirety for all purposes. The needle assembly 70 is loaded onto the needle arm 58, with the suture 78 extending parallel to the needle capture instrument 38 within the first lumen 34.

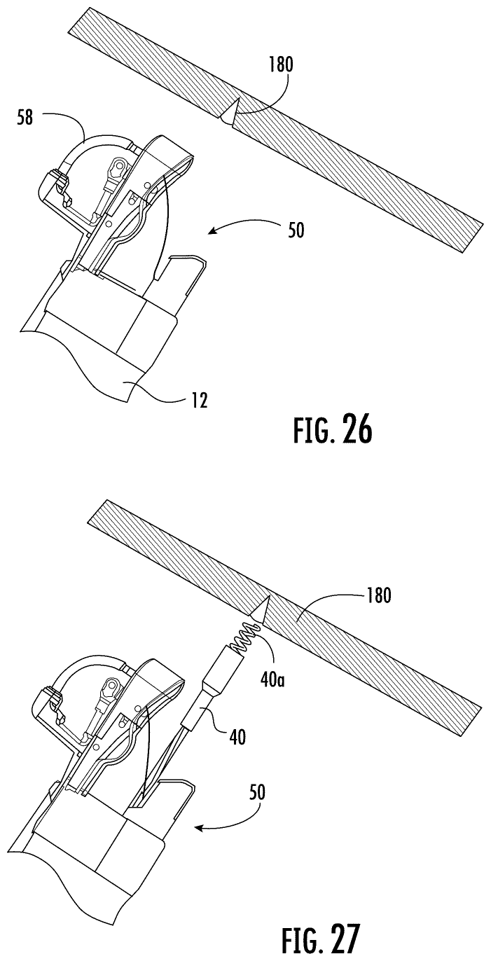

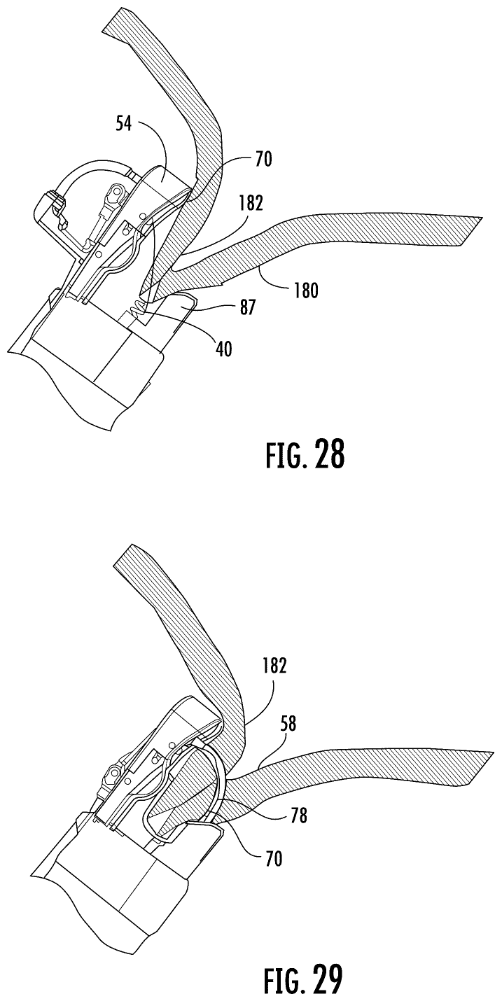

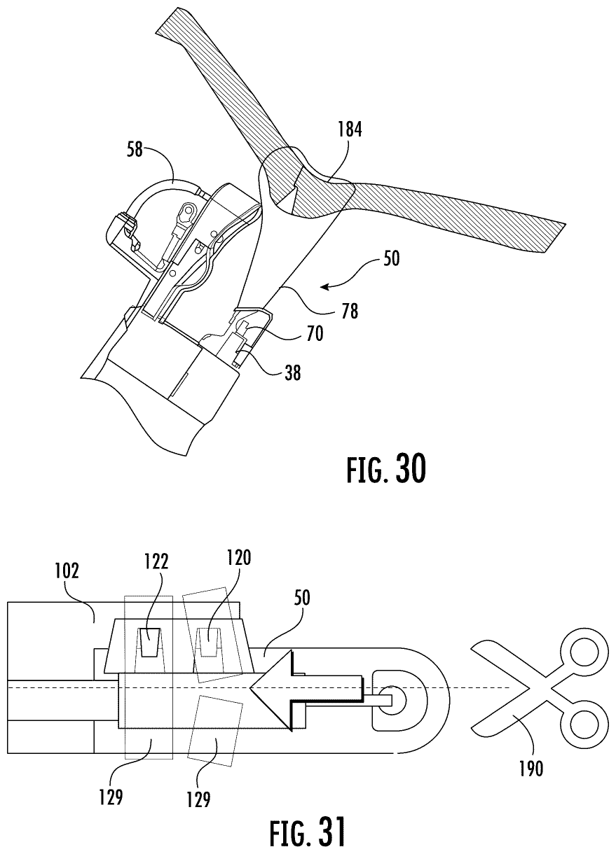

With reference to FIG. 2 and FIG. 26, the distal end of the endoscope 12 and cap assembly 50 of the suturing device 22 are advanced into a natural orifice of a patient, optionally through a guide tube (not shown), and approached to target tissue 180. If at any point it is necessary to reorient the endoscope 12 via retroflexion, the cap assembly 50 can be retroflexed as well, as described above, to aid and/or further direct the distal end of the endoscope and the cap assembly to the intended target tissue. Once adjacent the target tissue 180, the handle 24 of the suturing device 22 is operated to move the needle arm 58 into the open position, as shown in FIG. 25. Turning to FIG. 27, the end effector of the second device, e.g., a tissue retractor 40 having a helical coil 40a at its distal end, is advanced through the second port 32, into the second catheter 36 (FIG. 4) and out the second throughbore 88 (FIG. 5), and beyond the cap assembly 50. Suitable tissue retractor instruments are described in detail in U.S. Pat. No. 11,083,364, which patent is hereby incorporated herein by reference in its entirety for all purposes. Other tissue retractors, including forceps, may also be used. The helical coil 40a is operated to engage target tissue 180. The tissue retractor 40 is withdrawn to draw the tissue 180 against the tissue guard 87 and into a tissue fold 182 located within the path of the needle assembly 70; i.e., between the bracket 54 and needle guide 87, as shown in FIG. 28. The orientation of the second throughbore 88, either parallel or obliquely angled relatively to the first throughbore 86, is adapted to guide the tissue retractor to engage and retract tissue into the needle path. The handle 24 is then operated to move the needle arm 58 into the closed position, thereby piercing the tissue fold 182 and passing the needle assembly 70 with suture 78 through the tissue fold during the movement (FIG. 29). When the needle arm 58 is in the closed position, the needle is received within the distal end of the needle capture device 38 (FIG. 5). The needle capture device 38 is operated to securely engage the needle 70. The handle 24 is then operated to move the needle arm 58 toward the open position, thereby disengaging the needle arm 58 from the needle assembly 70, which remains in the needle capture device 38 (FIG. 30). The tissue retractor 40 is also released from the tissue and withdrawn back through the second catheter 36. The endoscope 12 is then moved to displace the cap assembly 50 relative to the sutured tissue 184. The needle 70 and suture 78 may be secured onto the tissue, such as by knotting or cinching, or the needle may be repositioned on the needle arm and additional suture loops may be formed within adjacent or other areas of tissue. Once the suturing is complete, the needle arm 58 is returned to a closed position, and the endoscope 12 and suturing device 22 are removed from the patient.

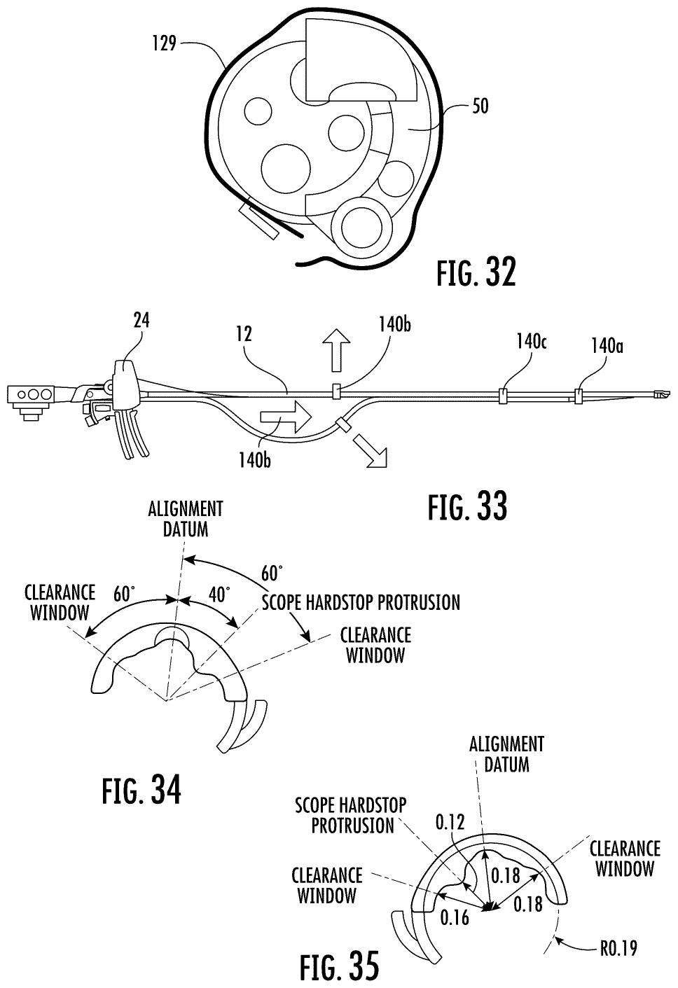

The suturing assembly is then released from over the endoscope by cutting, e.g., with a scissors 190, the two bands of tape 129 coupled to the hooks 120, 122 that secure the endoscope 12 relative to the cap assembly 50, and then unwrapping the tape 129 over the opening of the cap clip 102, as shown by both FIG. 31 and FIG. 32. Then, referring to FIG. 33, the tape strips 140b, 140c, 140a are broken apart, in turn, by applying force in the direction of arrow 174 to break each respective piece of tape at its perforation 152 (FIG. 14); as each tape strip was not directly adhered to the endoscope but only to itself and the sheath 89, the endoscope can be released once the strips are broken. The proximal handle 24 is then fully released from the endoscope 12.

In view of the above, in accordance with various principles of the present disclosure, an example of an embodiment of an endoscopic apparatus for use with an endoscope has a proximal handle assembly; a distal cap assembly; and a central portion adapted to extend along the endoscope between the handle assembly and the cap assembly. In some aspects, the handle assembly defines a recess in which the distal end of the endoscope is received. In some embodiments, the handle includes at least one hook at one side of the recess for receiving a tensile member that wraps around a portion of the cap assembly and the endoscope.

In some embodiments, the at least one hook is identified with a contrasting color relative to a majority of the cap assembly.

Additionally or alternatively, in some embodiments, the distal cap assembly includes a distal stop against which the distal end of the endoscope is intended to be abutted. In some embodiments, the distal stop includes indicia relative to which a feature of the endoscope is intended to be rotationally aligned. In some embodiments, the indicia include a contrasting color relative to a majority of the cap assembly. In some embodiments the indicia on the distal stop and the at least one hook are identified with a common color. In some embodiments, the feature of the endoscope with which the indicia are to be aligned is an instrument channel of the endoscope.

Additionally or alternatively, in some embodiments, the cap assembly is a suturing assembly including a movably mounted needle holder. In some aspects, operation of the proximal handle assembly moves the needle holder on the cap assembly.

In accordance with various principles of the present disclosure, an example of an embodiment of an endoscopic apparatus for use with an endoscope includes a proximal handle assembly; a distal cap assembly; and a central portion adapted to extend along the endoscope between the handle assembly and the cap assembly. In some aspects, the cap assembly defines a recess in which the distal end of the endoscope is received. Additionally or alternatively, the cap assembly includes two longitudinally spaced apart hooks at one side of the recess.

Additionally or alternatively, in some embodiments, each of the hooks are identified by a contrasting color relative to a color of a majority of the cap assembly.

Additionally or alternatively, in some embodiments, each of the hooks are identified by different hook indicia. In some embodiments, the hook indicia indicate an order of operation in which the hooks are intended to be used. In some embodiments, the hook indicia include numbers.

Additionally or alternatively, in some embodiments, the distal cap assembly includes a resilient clip that defines the recess.

Additionally or alternatively, in some embodiments the distal cap assembly includes a distal stop against which the distal end of the endoscope is intended to be abutted, the stop including contrasting alignment indicia relative to which a feature of the endoscope is intended to be rotationally aligned.

Additionally or alternatively, in some embodiments the distal cap assembly includes a distal stop against which the distal end of the endoscope is intended to be abutted, the stop including contrasting alignment indicia relative to which an instrument channel of the endoscope is intended to be rotationally aligned. In some embodiments the distal stop includes first and second recesses, and the alignment indicia is provided at the first recess.

In some embodiments, the cap assembly is a suturing assembly including a movably mounted needle holder, and operation of the proximal handle assembly moves the needle holder on the cap assembly.

In accordance with various principles of the present disclosure, an example of an embodiment of a securing system for use with an endoscope includes an endoscopic apparatus including a proximal handle assembly, a distal cap assembly; a central portion adapted to extend along the endoscope between the handle assembly and the cap assembly; and a spool of tape. In some embodiments, the distal cap assembly defines a recess in which the distal end of the endoscope is received and a hook at a side of the recess. In some embodiments, the spool of tape has a free end provided with a preformed loop adapted to be received over the hook. In some embodiments, the loop is adapted to be attached over the hook. In some embodiments, the tape is adapted to be wrapped about a portion of the cap assembly and the endoscope to couple the cap assembly and endoscope relative to each other.

In some embodiments, the system further includes a tape applicator including a handle and an extension with a spool mount. In some embodiments, the spool is coupled to the spool mount. In some embodiments, the spool is removably coupled to the spool mount.

In some embodiments, the cap assembly includes at least two longitudinally displaced hooks. In some embodiments, the securing system includes at least two spools of tape.

In some embodiments, the hook is identified by a contrasting color from a color of a majority of the cap assembly.

In some embodiments, the cap assembly includes at least two hooks. In some embodiments, each of the hooks is identified by different hook indicia. In some embodiments, the hook indicia identify an order of operation in which the hooks are intended to be used. In some embodiments, the hook indicia include a contrasting color from a majority of the cap assembly.

In some embodiments, the cap assembly is a suturing assembly including a movably mounted needle holder. In some embodiments, operation of the proximal handle assembly moves the needle holder on the cap assembly.

In accordance with various principles of the present disclosure, a method includes coupling a cap assembly of an endoscopic apparatus to an endoscope having a distal end. In some aspects, the cap assembly defines a recess in which the distal end of the endoscope is received. Additionally or alternatively, the cap assembly has first and second longitudinally displaced hooks at one side of the recess. In accordance with various principles of the present disclosure, the method includes inserting the distal end of the endoscope into the recess; providing a first length of tape having a first loop at a free end; coupling the first loop to the first hook; and wrapping at least a portion of the first length of tape about the cap assembly and the distal end of the endoscope.

In some aspects, the method further includes providing a second length of tape having a second loop at a free end; coupling the second loop to the second hook; and wrapping at least a portion of the second length of tape about the cap assembly and the distal end of the endoscope. In some aspects, the first hook is distally displaced from the second hook. In some aspects, the endoscopic apparatus includes a proximal handle and a central longitudinal sheath portion extending between the handle and the cap assembly. In some aspects, the central longitudinal portion includes a concave recess along its length. In some aspects, the method further includes inserting the endoscope into the recess of the central longitudinal sheath portion; and securing the central longitudinal sheath portion to the endoscope. In some aspects, the sheath portion is secured to the endoscope with a plurality of longitudinally spaced apart pieces of tape positioned at least partially circumferentially about the sheath portion and the endoscope. In some aspects, the central longitudinal sheath portion is crescent shaped and defines at least one lumen. In some aspects, at least one lumen includes a first lumen through which a flexible first catheter extends to the cap assembly, and a second lumen through which a flexible second catheter extends to the cap assembly. In some aspects, the cap assembly includes an actuation arm movably mounted thereon. In some aspects, the central longitudinal sheath portion includes a third lumen through which an actuation assembly extends. In some aspects, the actuation assembly is coupled between the proximal handle and the actuation arm and adapted for moving the actuation arm on the cap assembly upon operation of the proximal handle. In some aspects, the endoscopic apparatus includes a pull string having a proximal end and a distal end, the pull string extending from the proximal handle, through the central longitudinal sheath portion, and fixed to the cap assembly. In some aspects, the proximal end of the pull string is retracted relative to the proximal handle, and the distal end of the pull string is tensioned to cause the first and second catheters to flex and draw the cap assembly into retroflexion.

In some aspects, the cap assembly is a suturing assembly including a movably mounted needle holder.

In accordance with various principles of the present disclosure, an example of an embodiment of an endoscopic apparatus for use with an endoscope includes a proximal handle assembly; a distal cap assembly; a sheath portion; and a securing system. In some aspects, the cap assembly defines an assembly for positioning adjacent the distal end of the endoscope. In some aspects, the sheath portion defines a longitudinal recess. In some aspects, the sheath portion is adapted to extend along a portion of an exterior of the endoscope between the handle assembly and the cap assembly. In some aspects, the sheath portion is adapted to extend at least partially about the endoscope at the recess. In some aspects, the recess has a first side and a second side. In some aspects, the securing system is configured to secure the central portion to the exterior of the endoscope during in vivo operation of the endoscope. In some aspects, the securing system includes a plurality of longitudinally spaced apart lengths of tape pre-applied to the sheath portion on the first side of the recess. In some aspects, at least some of or each of the lengths of tape has an adhesive first portion at which the tape is adhered to the sheath on the first side of the recess, and a non-adhesive second portion extending from the first portion. In some aspects, at least some of or each of the lengths of tape has an adhesive third portion extending from the second portion and terminating in a free end.

In some aspects, the securing system further includes a removable non-adhesive protective strip over the third portion.

In some aspects, the protective strip extends into a tab with a free end protruding between the free end and the first portion.

Additionally or alternatively, the protective strip extends into an L-shaped non-adhesive tab. Additionally or alternatively, the tab has indicia indicating a direction in which the tab should be pulled to expose the third portion.

Additionally or alternatively, the protective strip has a contrasting color to the sheath portion.

In some aspects, each length of tape has a perforation between the, at, or near a junction of the second and third portions of the tape.

In some aspects, the securing system includes a first length of tape pre-applied at a distal end of the sheath portion, a second length of tape pre-applied at a central portion of the sheath portion, and a third length of tape pre-applied to the sheath portion between the first and second lengths of tape.

In some aspects, the sheath portion defines at least one lumen in a wall of the sheath portion.

In some aspects, the cap assembly is a suturing assembly including a movably mounted needle holder. In some aspects, operation of the proximal handle assembly moves the needle holder on the cap assembly.

In accordance with various principles of the present disclosure, a method of securing an endoscopic apparatus to an endoscope includes securing an endoscope having an external sheath to an endoscope. In some aspects, the endoscope has a circumference and a first length, and the sheath has a second length configured to extend about at least a portion of the circumference of the endoscope along at least a portion of the first length of the endoscope. In some aspects, the method includes providing the sheath with pre-applied portions of tape along second length; extending the sheath about the portion of the circumference of the endoscope along the first length; and wrapping the pre-applied portions of tape about a periphery of the endoscope and sheath to secure the sheath to the endoscope.

In some aspects, at least one or each pre-applied portion of tape includes an adhesive first portion at which the portion of tape is adhered to the sheath. Additionally or alternatively, at least one or each pre-applied portion of tape includes a non-adhesive second portion extending from the first portion. Additionally or alternatively, at least one or each pre-applied portion of tape includes an adhesive third portion extending from the second portion and terminating in a free end. Additionally or alternatively, at least one or each pre-applied portion of tape includes a removable protective strip over the third portion. In some aspects, the method further includes removing the removable protective strip from other the third portion, extending the second portion into contact with the endoscope, and adhering the third portion into contact with the sheath on an opposite side endoscope relative to the first portion.

In some aspects, the sheath defines a recess, the endoscope is inserted into the recess, and the first and third portions of the tape are located on opposite sides of the recess.

In some aspects, each pre-applied portion of tape includes a perforation. In some aspects, the method further includes releasing the endoscope from the sheath by applying a force to break the tape at the perforation. In some aspects, the tape is broken without cutting. In some aspects, the tape is broken by applying a force parallel to a longitudinal axis of the endoscope. In some aspects, the cap assembly is a suturing assembly including a movably mounted needle holder.

In accordance with various principles of the present disclosure, an example of an embodiment of an endoscopic apparatus for use with an endoscope includes a proximal handle assembly; a distal cap assembly; a sheath; a first catheter; a second catheter; and a transmission cable. In some aspects, the distal cap assembly is adapted to be positioned adjacent the distal end of the endoscope. In some aspects, the cap assembly has an end effector movable thereon. In some aspects, the sheath is a crescent-shaped flexible sheath. Additionally or alternatively, the sheath has a longitudinal side-opening recess sized to receive a portion of the endoscope between the handle assembly and the cap assembly. Additionally or alternatively, the sheath defines a first lumen, a second lumen, and a third lumen. In some aspects, the first catheter extends in and distally beyond the first lumen to the cap assembly. In some aspects, the second catheter extends in and distally beyond the second lumen to the cap assembly. In some aspects, the transmission cable extends from the handle assembly. In some aspects, the transmission assembly extends from the handle assembly into the third lumen. In some aspects, the transmission assembly extends distally beyond the third lumen to the cap assembly. In some aspects, the transmission assembly is operably coupled to the end effector. In some aspects, operation of the handle assembly moves the transmission cable to operate the end effector.

In some aspects, the cap assembly is a suturing assembly and the end effector is a needle holder.

In accordance with various principles of the present disclosure, an example of an embodiment of an endoscopic apparatus is configured for use with an endoscope having a proximal end and a distal end, an instrument channel, and a lens. In accordance with various principles of the present disclosure, the endoscopic apparatus includes a proximal handle; a distal cap assembly; and a central portion extending between the handle and cap assembly. In some aspects, the distal cap assembly defines an assembly for interacting with tissue. In some aspects, the distal cap assembly is adapted to be positioned adjacent the distal end of the endoscope. In some aspects, the distal cap assembly includes a clip to receive the distal end of the endoscope. In some aspects, the distal cap assembly includes a stop to limit distal travel of the endoscope. In some aspects, the stop has indicia against which a feature of the endoscope is rotationally aligned to ensure appropriate orientation of the lens and instrument channel of the endoscope without obstruction thereof. In some aspects, the central portion extends between the handle and cap assembly.

In some aspects, the stop is adapted to function with endoscopes from multiple manufacturers.

In some aspects, the cap assembly is a suturing assembly including a movably mounted needle holder. In some aspects, operation of the proximal handle moves the needle holder on the cap assembly.

In accordance with various principles of the present disclosure, an example of an embodiment of an endoscopic apparatus for use with an endoscope includes a proximal handle assembly; a distal cap assembly; a sheath; at least one catheter; and a pull string. In some aspects, the distal cap assembly is adapted to couple to the distal end of the endoscope spaced from the handle assembly by a first length. In some aspects, the sheath is a flexible sheath. In some aspects, the sheath has a proximal end and a distal end, the proximal end attached to the handle assembly. In some aspects, the sheath extends a second length less than the first length. In some aspects, the difference between the first length and the second length defines a gap. In some aspects, the sheath is adapted to extend at least partially about an exterior of the endoscope. In some aspects, the sheath defines at least one lumen. In some aspects, the at least one catheter extends through the at least one lumen from the proximal end of the sheath. In some aspects, the at least one catheter extends beyond the distal end of the sheath. In some aspects, the at least one catheter is fixed to the cap assembly. In some aspects, the pull string has a proximal end and a distal end. In some aspects, the pull string extends from the proximal end of the sheath, through the at least one lumen, out of the distal end of the sheath. In some aspects, the distal end of the pull string is fixed to the cap assembly. In some aspects, the proximal end of the pull string extends from an opening in the handle assembly. In some aspects, when the proximal end of the pull string is retracted relative to the handle, the distal end of the pull string is tensioned to cause the catheter to flex and draw the cap assembly into retroflexion across the gap.

In some aspects, the distal cap assembly is a suturing assembly including a needle movable relative to the at least one catheter. In some aspects, the pull string extends through a common lumen with a transmission cable for operating the suturing assembly.

In some aspects, the apparatus includes a pull handle attached to the proximal end of the pull string. In some aspects, the proximal handle assembly is adapted to releasably store the pull handle.

In some aspects, the apparatus further includes a locking system to temporarily retain the pull string under tension. In some aspects, the proximal handle assembly includes the locking system. In some aspects, the locking system includes a post having a seat and a washer provided over the post on the seat. In some aspects, the proximal end of the pull string is retained in position on the post. In some aspects, the proximal end of the pull string is retained in position on the post when wrapped around the post between the seat and the washer. In some aspects, the washer is made of a resilient material.

In accordance with various principles of the present disclosure, an example of an embodiment of an endoscopic system includes an endoscope and an external instrument channel device couplable to the endoscope. In some aspects, the endoscope has a proximal end and a distal end and an outer surface extending between the proximal and distal ends. In some aspects, the proximal end has a first handle. In some aspects, the distal end is adapted to retroflex upon actuation of the proximal end. In some aspects, the instrument channel device has a second handle. In some aspects, the instrument channel device has a sheath adapted to be coupled in close proximity to the outer surface of the endoscope between the proximal and distal ends. In some aspects, the instrument channel device has a cap removably coupled to the distal end of the endoscope. In some aspects, the instrument channel device has an instrument channel extending from the distal end of the sheath to the cap. In some aspects, the instrument channel device has a pull string extending from the second handle, through the sheath, and fixed relative to the cap. In some aspects, when the pull string is placed under tension, the cap is biased into a retroflex position about the instrument channel.

In some aspects, the cap is part of a suturing assembly.

In some aspects, the system further includes a pull handle attached to the proximal end of the pull string. In some aspects, the external instrument channel device includes a proximal handle assembly. In some aspects, the proximal handle assembly is adapted to releasably store the pull handle.

In some aspects, the system further includes a locking system to temporarily retain the pull string under tension. In some aspects, the external instrument channel device includes a proximal handle assembly, and the proximal handle assembly is provided with the locking system. In some aspects, the locking system includes a post having a seat and a washer provided over the post on the seat. In some aspects, the proximal end of the pull string is retained in position on the post. In some aspects, the proximal end of the pull string is retained in position on the post when wrapped around the post between the seat and the washer.

The suturing assemblies described above are adapted for use with an endoscope that does not necessarily have at least two instrument channels. As such, the suturing system can be used with smaller endoscopes that are available in many surgical settings and which can be more easily advanced through a natural orifice. In addition, as indicated aspects of the system can be used in other surgical treatment settings other than for suturing.

There have been described and illustrated herein embodiments of a suturing system as well as a surgical treatment system, as well as methods of using the same. While particular embodiments of the invention have been described, it is not intended that the invention be limited thereto, as it is intended that the invention be as broad in scope as the art will allow and that the specification be read likewise. Thus, while particular instruments and devices for advancement through the first and second lumen have been disclosed, it will be appreciated that other instruments can also be used through such lumen for like or even different purpose. Also, while the treatment system has been particularly described with respect to a cap assembly having an end effector in the form of a needle arm that carries a needle, it is recognized that alternatively one or more movable end effectors with other structure and purpose can be provided to the cap assembly. Also, while a tissue anchor in the form of a needle assembly has been described, the end effector can deploy different types of tissue anchors, including, e.g., clips. In addition, while a particular needle assembly has been described, other needle assemblies can similarly be used. Also, the size and instrument channel features of the endoscope with which the system is used is not critical, it is appreciated that various prior art systems cannot be properly used in a suturing operation in conjunction with endoscopes having fewer than two instrument channels, one for receiving a needle exchange device and the other for receiving a tissue retractor, whereas the present system is capable of complete operation without the provision of any channels through the endoscope. Further, while it is indicated that various features described herein are not limited to suturing applications, such as the retroflex system, it is specifically recognized that the retroflex system may be used in association with a cap assembly adapted to provide various other surgical applications including, but not limited to, staplers, clip appliers, band ligators, tissue manipulating instruments, cutting instruments, forceps, biopsy instruments, injection devices, as well as cap assemblies that have no significant function other than to support external catheters for passthrough of instruments. It will therefore be appreciated by those skilled in the art that yet other modifications could be made to the provided invention without deviating from its scope as claimed.

It is to be understood by one of ordinary skill in the art that the present discussion is a description of illustrative examples of embodiments only, and is not intended as limiting the broader aspects of the present disclosure.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims. Various further benefits of the various aspects, features, components, and structures of devices and systems such as described above, in addition to those discussed above, may be appreciated by those of ordinary skill in the art.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A retroflexion device for retroflexing a flexible elongate member of a medical device, said retroflexion device comprising:
a distal mount configured to be mounted with respect to the distal end of the flexible elongate member;
a pivot mount configured to be mounted with respect to the flexible elongate member proximal to the distal mount; and
an elongated actuation component having a proximal end and a distal end with a length extended therebetween selected to extend along the length of the flexible elongate member;
wherein:
a proximal end of said actuation component is controllable along a proximal end of the flexible elongate member;
a distal end of said actuation component is operatively associated with a distal end of the flexible elongate member via said distal mount; and
an actuation region of said actuation component pivots with respect to said pivot mount when said actuation component is pulled proximally to cause the flexible elongate member to retroflex.

2. The retroflexion device of claim 1, further comprising a flexible tubular elongate member having at least one lumen extending therethrough through which said actuation component extends.

3. The retroflexion device of claim 2, wherein said flexible tubular elongate member is configured to be mounted along the flexible elongate member.

4. The retroflexion device of claim 3, wherein:
said flexible tubular elongate member has a proximal end and a distal end defining a length therebetween selected to be less than the length of the flexible elongate member;
said flexible tubular elongate member is configured to be mounted with respect to the flexible elongate member with the distal end of the flexible tubular elongate member proximal to the distal end of the flexible elongate member; and
the distal end of said flexible tubular elongate member forms said pivot mount for said actuation component.

5. The retroflexion device of claim 2, wherein said flexible tubular elongate member has more than one lumen extending therethrough to allow additional devices to be extended through said flexible tubular elongate member.

6. The retroflexion device of claim 2, wherein:
said flexible tubular elongate member has a proximal end and a distal end; and
said retroflexion device further comprises a proximal mount provided along the proximal end of said flexible tubular elongate member; and
the proximal end of said actuation component is operatively associated with said proximal mount.

7. The retroflexion device of claim 1, further comprising a proximal mount, the proximal end of said actuation component being operatively associated with said proximal mount.

8. The retroflexion device of claim 7, further comprising a pull handle on the proximal end of said actuation component.

9. The retroflexion device of claim 8, wherein said proximal mount includes a pull handle mount.

10. The retroflexion device of claim 7, further comprising a locking system mounted on said proximal mount and configured to temporarily retain said actuation component under tension to retroflex said flexible elongate member.

11. A retroflexion system comprising:
a medical device with a flexible elongate member having a proximal end and a distal end and an outer surface extending between the proximal end and the distal end;
a flexible tubular elongate member having a proximal end and a distal end and defining a lumen therethrough, said flexible tubular elongate member configured to be mounted with respect to the outer surface of said flexible elongate member; and
an actuation component extending through the lumen through said flexible tubular elongate member and having a distal end operatively associated with the distal end of said flexible elongate member, and a proximal end accessible to pull said actuation component proximally to retroflex the distal end of said flexible elongate member.

12. The retroflexion system of claim 11, further comprising:
a distal mount coupled to the distal end of said flexible elongate member, the distal end of said actuation component operatively associated with the distal end of said flexible elongate member via said distal mount; and
a pivot mount mounted with respect to said flexible elongate member proximal to said distal mount and about which an actuation region of said actuation component pivots to retroflex the distal end of said flexible elongate member.

13. The retroflexion system of claim 12, wherein:
the length of said flexible tubular elongate member is less than the length of said flexible elongate member;
said flexible tubular elongate member is mounted with respect to said flexible elongate member with the distal end of said flexible tubular elongate member proximal to the distal end of said flexible elongate member; and
the distal end of said flexible tubular elongate member defines said pivot mount.

14. The retroflexion system of claim 11, further comprising a proximal mount provided along the proximal end of said flexible elongate member and operatively associated with the proximal end of said flexible elongate member, wherein the proximal end of said actuation component is operatively associated with said proximal mount.

15. The retroflexion system of claim 14, further comprising a pull handle at a proximal end of said actuation component, wherein said proximal mount comprises a mount for said pull handle and a locking system configured to temporarily retain said actuation component under tension to retroflex said flexible elongate member.

16. The retroflexion system of claim 11, wherein said medical device is an endoscope.

17. A method of retroflexing a distal end of a flexible elongate member of a medical device, said method comprising:
extending an elongated actuation component along the exterior of the flexible elongate member and fixing a distal end of the actuation component with respect to a distal end of the flexible elongate member; and
pulling the actuation component proximally to cause the actuation component to pivot about a pivot mount proximal to the distal end of the actuation component and the distal end of the flexible elongate member to cause the flexible elongate member to retroflex.

18. The method of claim 17, wherein pulling the actuation component comprises pulling the proximal end of the actuation component adjacent a proximal end of the flexible elongate member.

19. The method of claim 18, further comprising securing a portion of the actuation component with respect to a locking system to hold the flexible elongate member in a retroflexed configuration.

20. The method of claim 17, further comprises retroflexing the distal end of the flexible elongate member greater than 180°.
